(19) United States
(12) Patent Application Publication
(10) Pub. No.: US 2025/0154197 A1
(43) Pub. Date: May 15, 2025

(54) LIPOLYTIC PEPTIDES

(71) Applicant: FUNDACIÓ HOSPITAL UNIVERSITARI VALL D'HEBRON - INSTITUT DE RECERCA, BARCELONA (ES)

(72) Inventors: DAVID MARTINEZ SELVA, BARCELONA (ES); RAFAEL SIMÓ CANONGE, SANT CUGAT DEL VALLÈS (ES)

(21) Appl. No.: 18/715,478

(22) PCT Filed: Dec. 2, 2022
(86) PCT No.: PCT/EP2022/084303

§ 371 Date: May 31, 2024

(30) Foreign Application Priority Data
Dec. 3, 2021 (EP) 21383114.2

(51) Int. Cl. C07K 7/06; A61K 8/64; A61Q 19/00

(52) U.S. Cl. CPC C07K 7/06; A61K 8/64; A61Q 19/00; A61K 2800/91

(57) ABSTRACT

Lipolytic peptides having a length from 5 to 9 amino acids and corresponding to SEQ ID NO: 54 or a salt thereof, wherein: X¹ represents W or N; X² represents P, N, a basic amino acid or a hydrophobic amino acid; X³ and X⁴ are the same or different and represent A, V, L or I; X⁵ represents F or P; subindexes “a”, “b”, “c”, “d”, “e”, “f” and “g” are integer numbers independently selected from 0 and 1, provided that two, three, four, five or six of the subindexes “a”, “b”, “c”, “d”, “e”, “f” and “g” represent 1 and the remaining represent “0”. Peptides of sequence SEQ ID NO: 39-46 or any salt thereof. The peptides show improved lipolytic activity when compared with other lipolytic agents, such as caffeine or hexa-39.

This application claims the benefit of European Patent Application no. EP21383114.2 filed on 3 Dec. 2021.

### TECHNICAL FIELD

The present invention refers to the field of peptides and lipolysis. In particular, the present invention provides lipolytic peptides, compositions comprising thereof and uses in therapy and cosmetics, in particular, as lipolytic agents, among others.

### BACKGROUND ART

The management of fats in the human body is regulated by the adipose tissue, and more particularly by the adipocytes which are the cells which compose this tissue. This regulation involves a plurality of cellular mechanisms, among which are mechanisms for the storage of fats, and mechanisms for the release, breakdown and/or transformation of fats.

In general, the metabolism of fat cells has a major effect on the overall metabolism of the human body. This excess is called overweight and can go as far as obesity. Today, around 30% of the population is overweight and obese.

The state of the art describes plants having an effect on adipose tissue.

In particular, coffee and tea have effects on fat tissue. The effects are mainly linked to the presence of caffeine in these plants. Caffeine, either of natural or synthetic origin, is notably involved in the activation of lipolysis and the general stimulation of the metabolism of adipocytes.

However, the daily dosage relating to caffeine is difficult to control. This is particularly due to the presence of this molecule in many foods. Accurate monitoring is difficult, if not impossible. In addition, beyond its effect on adipose tissue, caffeine has effects on other metabolic processes in humans. Generally, caffeine is known to be a stimulant and a psychostimulant. It has an effect on the cardiovascular system which can cause a harmful acceleration of the heart rate. In some cases, caffeine has a diuretic effect. It is also an antagonist of the receptors for the neurotransmitter adenosine in the central nervous system. Thus, caffeine can generate physical and psychological dependence in some people.

Apart from adrenergic substances (adrenaline and analogues) whose activity of stimulating lipolysis in adipocytes is well known but whose use in cosmetic products is prohibited, small peptides have also been developed. For instance, document WO2012130775 discloses small peptides modulating the expression of the coactivator PGC-Ict. Among these peptides, the one known as “acetyl-hexapeptide-39” is disclosed.

In spite of the efforts made, however, there is still the need of small peptides with an improved profile, easily synthesisable and safer.

### SUMMARY OF THE INVENTION

The present inventors have developed short peptides, having from 5 to 9 amino acids, with a remarkable lipolytic activity when tested in vitro.

As it has been explained above, caffeine is one of the most widely used lipolytic agents. However, caffeine is a stimulant and it exerts some negative effects on some metabolic processes such as a harmful acceleration of the heart rate or dependency.

The present inventors have found that the peptides of the invention, even having such short length, are surprisingly more active than caffeine: as it is shown in Table 6a below, the peptides of the invention show a remarkable increase of the lipolytic activity at concentrations equal or remarkably lower (i.e., 2-fold, 10-fold or even 100-fold lower) than the one of caffeine.

The above was found out with mice adipocytes. But it was also confirmed using human adipocytes as illustrated in Table 6b.

The present inventors have also found that the D-decapeptide of sequence SEQ ID NO: 3, optionally with the N- and/or C-terminal derivatized (sequences SEQ ID NO: 4 to 6), has a remarkable lipolytic activity. The inventors found that this D-decapeptide showed an increase in lipolytic activity of about 2-fold, when compared to the L version. This finding was surprising because the test conditions followed to determine the lipolytic activity allowed the inventors to conclude that such remarkable improved activity was not due to a “stability” effect (no test conditions are used to affect such property).

Not only that, but also, the present inventors have surprisingly found the D-decapeptide of sequence SEQ ID NO: 3 provides such improved lipolytic activity remarkably earlier, when compared with the corresponding L-version (SEQ ID No: 2). As it can be derived from Table 7 below, the peptide of the invention provided at 120′ a significant amount of glycerol, indicative of the lipolysis effect. On the contrary, neither caffeine nor the comparative peptide (L-version), at the same dose, provided any significant activity at that time. On the other hand, Table 7 emphasizes again the improved efficiency in inducing lipolysis when the peptides of the invention are used, even after a very short period of time after the administration. In this case, peptide SEQ ID NO: 3 was found to be more than 24-fold more efficient at time 120′ vs caffeine or SEQ ID NO:2 and more than 5-fold at 240′.

In conclusion, the peptides of the invention not only are more efficient in providing a lipolytic activity, but also (1) induce the onset of the lipolytic activity remarkably earlier, and (2) prolong the lipolytic effect for longer periods of time.

Furthermore, the small size of the peptides of the invention facilitates their synthesis at industrial scale.

Advantageously, the peptides of the invention, due to such improved properties, can be used at low doses, avoiding all or part of the negative effects related to the use of high doses of caffeine.

Thus, in one aspect the present invention provides lipolytic peptide which:
(a) has a sequence length from 5 to 9 amino acids, each amino acid being either L- or D-amino acid, the peptide corresponding to a sequence SEQ ID NO: 54 or an acceptable salt thereof:

(T)ₐ(X¹)_{b}D(X²)_{g}EG(X³)_{c}(X⁴)_{d}(X⁵)ₑ(Y)_{f}

wherein:
X¹ represents W, N, Q or K;
X² represents P, N, a basic amino acid or a hydrophobic amino acid
X³ and X⁴ are the same or different and represent A, V, L or I;
X⁵ represents F or P,
subindexes “a”, “b”, “c”, “d”, “e”, “f” and “g” are integer numbers independently selected from 0 and 1; provided that two, three, four, five or six of the subindexes “a”, “b”, “c”, “d”, “e”, “f” and “g” represent 1; and the remaining subindexes represent zero;
   or, alternatively,
(b) is selected from the group consisting of: SEQ ID NO: 39-46, and any salt thereof;
and wherein
the N-terminal end of the lipolytic peptide corresponds to —NR₁R₂, being R₁ and R₂ independently selected from the group consisting of —H, —C(O) R₃, a triglyceride and (C₁-C₂₀)alkyl;
the C-terminal end of the lipolytic peptide corresponds to —COOR₄ and —C(O) NR₅R₆; and
R₃ to R₆ are radicals independently selected from the group consisting of:—H and (C₁-C₂₀)alkyl.

The data provided below in Table 6b also illustrates that the short peptides of the invention are highly efficient when compared to hexapeptide-39 (SEQ ID NO: 53). In particular, it was found that they provided an increase in the lipolytic effect on human adipocytes of at least about 40% vs hexapeptide-39.

In a second aspect, the present invention provides a cosmetic composition comprising a cosmetically effective amount of a peptide or salt thereof, as defined in the first aspect of the invention, together with one or more cosmetically acceptable carriers or excipients.

These compositions of the present invention may also provide additional benefits, including stability, absence of significant (consumer-unacceptable) skin irritation, good aesthetics, or improved skin diffusion, among others.

In a third aspect the present invention provides a pharmaceutical composition comprising a therapeutically effective amount of a peptide or salt thereof, as defined in the first aspect of the invention, together with one or more therapeutically acceptable carriers or excipients.

In a fourth aspect the present invention provides a kit of parts comprising:
(a) a peptide as defined in the first aspect of the invention or a composition as defined in the second or third aspect of the invention, and
(b) a device selected from the group consisting of: an injection device, such as a syringe, one or more micro-needles, an implant, or a biodegradable container, said kit comprising the peptide or composition physically separated from the device.

In a fifth aspect, the present invention provides the use of the peptide as defined in the first aspect of the invention as lipolytic agent.

The peptides of the invention, due to its role as lipolytic agents, can be used to reduce the lipids (mass of adipose tissue) that accumulate in many parts of the animal body. In the particular case of the human body, the lipid accumulation (adipose tissue with high amounts of triglycerides) tends to accumulate in the hips, waist, abdomen, and thighs.

In a sixth aspect, the present invention provides the use of the peptide as defined in the first aspect of the invention or of the cosmetic composition as defined in the second aspect of the invention, in cosmetics.

In a seventh aspect, the present invention provides the use of a cosmetically effective amount of the peptide as defined in the first aspect of the invention or of the cosmetic composition as defined in the second aspect of the invention in improving the bodily appearance of a mammal with subcutaneous fat herniated or accumulated within the fibrous connective tissue under the skin. This aspect can also be alternatively formulated as a cosmetic method for improving the bodily appearance of a mammal with subcutaneous fat herniated or accumulated within the fibrous connective tissue under the skin, the method comprising applying, particularly topically or intra-dermally applying, the peptide as defined in the first aspect of the invention or the cosmetic composition as defined in the second aspect of the invention, to the subject.

In a further aspect, the present invention provides the peptide as defined in the first aspect of the invention or the pharmaceutical composition of the third aspect of the invention, for use in therapy.

In a final aspect the present invention provides the peptide as defined in the first aspect of the invention or the pharmaceutical composition of the third aspect of the invention for use in the treatment of the accumulation of fat in an internal organ. This aspect can be formulated as the use of a peptide as defined in the first aspect of the invention or the pharmaceutical composition of the third aspect of the invention for the manufacturing of a medicament for the treatment of the accumulation of fat in an internal organ. This aspect can also be formulated as a method for treating the accumulation of fat in an internal organ, the method comprising administering a therapeutically effective amount of a peptide as defined in the first aspect of the invention or the pharmaceutical composition of the third aspect of the invention, to a subject in need thereof.

### DETAILED DESCRIPTION OF THE INVENTION

All the terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

For the purposes of the present invention, any ranges given include both the lower and the upper end-points of the range.

The present inventors provide peptides or salts thereof as provided in the first aspect of the invention, which are useful as lipolytic agents.

In the present invention, the term “amino acid” refers to a molecule containing both an amino group and a carboxyl group.

Basic amino acids (hereinafter also referred as “basic and polar amino acid”) have side chains containing nitrogen and resemble ammonia which is a base (such as amines, guanidines, or imidazole). Their pKa's are high enough that they tend to bind protons, gaining a positive charge in the process. Illustrative non-limitative examples of basic amino acids are Lys, Arg, and His.

The term “Hydrophobic amino acids”, as used herein, refers to amino acids having hydrophobic side chains, which tend to be repelled from water. When part of a protein, these amino acids typically form the hydrophobic core of proteins, which is isolated from the polar solvent. Non-limitative examples of hydrophobic amino acids include glycine (Gly), alanine (Ala), valine (Val), leucine (Leu), isoleucine (Ile), proline (Pro), phenylalanine (Phe), methionine (Met), and tryptophan (Trp). In a particular embodiment, the hydrophobic amino acids referred herein are selected from the list consisting of glycine (Gly), alanine (Ala), valine (Val), leucine (Leu), isoleucine (Ile), proline (Pro), phenylalanine (Phe), methionine (Met), and tryptophan (Trp). In another embodiment, the hydrophobic amino acids referred herein are selected from the list consisting of alanine (Ala), valine (Val), leucine (Leu), isoleucine (Ile), proline (Pro), phenylalanine (Phe) and cysteine (Cys). In a particular embodiment, the hydrophobic amino acids referred herein are selected from the list consisting of alanine (Ala), valine (Val), and leucine (Leu). Amino acids used in the construction of peptides of the present invention may be prepared by organic synthesis, or obtained by other routes, such as, for example, degradation of or isolation from a natural source.

As used herein, the term “acceptable salt” or “salt” refers to those salts which are, within the scope of sound medical judgment, suitable for use in contact with the tissues of humans and lower animals without undue toxicity, irritation, allergic response and the like, and are commensurate with a reasonable benefit/risk ratio. Appropriate acceptable salts are well known in the art. Examples of pharmaceutical acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other acceptable salts include adipate, alginate, ascorbate, aspartate, benzenesulfonate, benzoate, bisulphate, borate, butyrate, camphorate, camphorsulfonate, citrate, cyclopentanepropionate, digluconate, dodecylsulphate, ethanesulfonate, formate, fumarate, glucoheptonate, glycerophosphate, gluconate, hemisulphate, heptanoate, hexanoate, hydroiodide, 2-hydroxy-ethanesulfonate, lactobionate, lactate, laurate, lauryl sulphate, malate, maleate, malonate, methanesulfonate, 2-naphthalenesulfonate, nicotinate, nitrate, oleate, oxalate, palmitate, pamoate, pectinate, persulphate, 3-phenylpropionate, phosphate, picrate, pivalate, propionate, stearate, succinate, sulphate, tartrate, thiocyanate, p-toluenesulfonate, undecanoate, valerate salts, and the like. Salts derived from appropriate bases include alkali metal, alkaline earth metal, and ammonium. Representative alkali or alkaline earth metal salts include sodium, lithium, potassium, calcium, magnesium, and the like. Further acceptable salts include, when appropriate, nontoxic ammonium, quaternary ammonium, and amine cations formed using counterions such as halide, hydroxide, carboxylate, sulphate, phosphate, nitrate, lower alkyl sulfonate and aryl sulfonate.

The term (C₁-C₂₀)alkyl refers to a saturated straight or branched alkyl chain having from 1 to 20 carbon atoms. Concomitantly, the terms (C₁-C₁₅)alkyl and (C₁-C₁₀)alkyl refer to saturated straight or branched alkyl chains having each from 1 to 15 carbon atoms, or 1 to 10 carbon atoms, respectively.

The term “D amino acid”, “D-aa” or “Daa” (wherein the amino acid (aa) can be any amino acid referred herein), refers to an amino acid having a D configuration, well known by an expert in the field.

The term “L amino acid”, “L-aa” or “Laa” (wherein the amino acid (aa) can be any amino acid referred herein), refers to an amino acid having an L configuration, well known by an expert in the filed.

Unless otherwise stated, the one or more of the amino acids forming the peptides of the invention can have L- or D-configuration.

In one embodiment of the first aspect of the invention, optionally in combination with any of the embodiments provided above or below, at least one of the amino acids forming the peptide of sequence SEQ ID NO: 54 is a D-amino acid. In another embodiment of the first aspect of the invention, optionally in combination with any of the embodiments provided above or below, 1, 2, 3, 4, 5, 6, 7, 8, 9 or all the amino acids forming the peptide of sequence SEQ ID NO: 54 are D-amino acids. In another embodiment of the first aspect of the invention, optionally in combination with any of the embodiments provided above or below, all the amino acids forming the peptide of sequence SEQ ID NO: 54 are D-amino acids. In an embodiment of the first aspect of the invention, those amino acids that are not D-amino acids are L-amino acids in the peptide of the first aspect of the invention, preferably in the peptide with SEQ ID NO:54. In another embodiment of the first aspect of the invention, optionally in combination with any of the embodiments provided above or below, all the amino acids forming the peptide of sequence SEQ ID NO: 54 are L-amino acids.

In one embodiment of the first aspect of the invention, optionally in combination with any of the embodiments provided above or below, at least one of the amino acids forming the peptide of sequence SEQ ID NO: 1, is a D-amino acid. In another embodiment of the first or second aspect of the invention, optionally in combination with any of the embodiments provided above or below, 1, 2, 3, 4, 5, 6, 7, 8, 9 or all the amino acids forming the peptide of sequence SEQ ID NO: 1 are D-amino acids. In another embodiment of the first or second aspect of the invention, optionally in combination with any of the embodiments provided above or below, all the amino acids forming the peptide of sequence SEQ ID NO: 1 are D-amino acids. In an embodiment of the first aspect of the invention, optionally in combination with any of the embodiments provided above or below, those amino acids that are not D-amino acids are L-amino acids in the peptide of SEQ ID NO: 1. In another embodiment of the first or second aspect of the invention, optionally in combination with any of the embodiments provided above or below, all the amino acids forming the peptide of sequence SEQ ID NO: 1 are L-amino acids.

In one embodiment of the first aspect of the invention the lipolytic peptide is one selected from sequences SEQ ID NO: 39 to 46 and salts thereof wherein 1, 2, 3, 4, 5, 6, 7, 8, 9 or all the amino acids are D-amino acids. In one embodiment of the first aspect of the invention the lipolytic peptide is one selected from sequences SEQ ID NO: 39 to 46 and salts thereof wherein all the amino acids are D-amino acids. In an embodiment of the first aspect of the invention, optionally in combination with any of the embodiments provided above or below, those amino acids that are not D-amino acids are L-amino acids in the peptide of sequences SEQ ID NO: 39 to 46. In another embodiment of the first or second aspect of the invention, optionally in combination with any of the embodiments provided above or below, all the amino acids forming the peptide of sequence SEQ ID NO: 43-46 are L-amino acids.

In an embodiment of the first aspect of the invention, optionally in combination with any of the embodiments provided above or below, 1, 2, 3, 4, 5, 6, 7, 8, 9 or all the amino acids of the lipolytic peptide of the invention are D-amino acids. In a particular embodiment of the first aspect of the invention, all the amino acids of the lipolytic peptide of the invention are D-amino acids.

In another embodiment of the first aspect of the invention, optionally in combination with any of the embodiments provided above or below, the lipolytic peptide is a peptide of sequence SEQ ID NO: 54 or a salt thereof, as defined above, wherein:
X² represents P, N, or a basic amino acid,
subindex “g” represents 1, and
X⁵ represents F,
   and said peptide corresponds to a peptide of sequence SEQ ID NO.1.

As well understood by an expert in the field, one, two, three, four or five of the subindexes “a”, “b”, “c”, “d”, “e”, “f” of the lipolytic peptide defined in the previous embodiment represent 1, and the remaining subindexes represent zero.

Therefore, in a particular embodiment of the first aspect of the invention, optionally in combination with any of the embodiments provided above or below, the lipolytic peptide having a sequence length from 5 to 9 amino acids, wherein each amino acid being either L- or D-amino acid, corresponds to a sequence SEQ ID NO: 1 or a salt thereof:

(T)ₐ(X¹)_{b}D(X²)EG(X³)_{c}(X⁴)_{d}(F)ₑ(Y)_{f}

wherein:
X¹ represents W, N, Q or K;
X² represents P, N or a basic amino acid;
X³ and X⁴ are the same or different and represent A, V, L or I;
   subindexes “a”, “b”, “c”, “d”, “e”, and “f” are integer numbers independently selected from 0 and 1; provided that one, two, three, four or five of the subindexes “a”, “b”, “c”, “d”, “e”, and “f” represent 1; and the remaining subindexes represent zero.

Therefore, in a particular embodiment of the first aspect of the invention, the lipolytic peptide of the invention:
(a) has a sequence length from 5 to 9 amino acids, each amino acid being either L- or D-amino acid, the peptide corresponding to sequence SEQ ID NO: 1 or an acceptable salt thereof:

(T)ₐ(X¹)_{b}D(X²)EG(X³)_{c}(X⁴)_{d}(F)ₑ(Y)_{f}

wherein:
X¹ represents W, N, Q or K;
X² represents P, N or a basic amino acid;
X³ and X⁴ are the same or different and represent A, V, L or I;
subindexes “a”, “b”, “c”, “d”, “e”, and “f” are integer numbers independently selected from 0 and 1; provided that one, two, three, four or five of the subindexes “a”, “b”, “c”, “d”, “e”, and “f” represent 1; and the remaining subindexes represent zero;
   5 or, alternatively,
(b) is selected from the group consisting of: SEQ ID NO: 39-46, and any salt thereof;
   and wherein
   the N-terminal end of the lipolytic peptide corresponds to —NR₁R₂, being R₁ and R₂ independently selected from the group consisting of —H, —C(O) R₃, a triglyceride and (C₁-C₂₀)alkyl;
   the C-terminal end of the lipolytic peptide corresponds to —COOR₄ and —C(O) NR₅R₆; and
   R₃ to R₆ are radicals independently selected from the group consisting of:—H and (C₁-C₂₀)alkyl.

In one embodiment of the first aspect of the invention, optionally in combination with any of the embodiments provided above or below, the lipolytic peptide is a peptide of sequence SEQ ID NO: 1 or a salt thereof, as defined above, wherein X¹ represents W or N; particularly X¹ represents a D-amino acid selected from D-W or D-N. In a particular embodiment, the lipolytic peptide is a peptide of sequence SEQ ID NO: 1 or a salt thereof, as defined above, wherein X¹ represents W, particularly X¹ represents a D-amino acid and is D-W. In another particular embodiment, the lipolytic peptide is a peptide of sequence SEQ ID NO: 1 or a salt thereof, as defined above, wherein X¹ represents N, particularly X¹ represents a D-amino acid and is D-N.

In one embodiment of the first aspect of the invention, optionally in combination with any of the embodiments provided above or below, the lipolytic peptide is a peptide of sequence SEQ ID NO: 1 or a salt thereof, as defined above, wherein X² represents P or N; particularly X² represents a D-amino acid selected from D-P or D-N.

In one embodiment of the first aspect of the invention, optionally in combination with any of the embodiments provided above or below, the lipolytic peptide is a peptide of sequence SEQ ID NO: 1 or a salt thereof, as defined above, wherein X³ and X⁴ are the same or different and represent V, L or I; particularly X³ and X⁴ are D-amino acids, the same or different, and represent D-V, D-L or D-I. In another embodiment of the first aspect of the invention, the lipolytic peptide is a peptide of sequence SEQ ID NO: 1 or a salt thereof, as defined above, wherein X³ represents V, L or I, particularly X³ represents a D-amino acid selected from D-V, D-L and D-I. In another embodiment of the first aspect of the invention, the lipolytic peptide is a peptide of sequence SEQ ID NO: 1 or a salt thereof, as defined above, wherein X⁴ represents V, L or I, particularly X⁴ represents a D-amino acid selected from D-V, D-L and D-I.

In one embodiment of the first aspect of the invention, optionally in combination with any of the embodiments provided above or below, the lipolytic peptide is a peptide of sequence SEQ ID NO: 1 or a salt thereof, made of L- and/or D-amino acids, as defined above, which is selected from the group consisting of sequences SEQ ID NO: 47 to SEQ ID NO: 49 and any salt thereof:
SEQ ID NO: 47 (T) a (X¹)_{b}DPEGV (X⁴). (F) e (Y) f, wherein “a”, “b”, “d”, “e”, and “f” are as defined in claim 1, X¹ represents W or N, and X⁴ represents I or L;
SEQ ID NO: 48 (T) a (X¹)_{b}DPEGX³ (F): (Y) f, wherein “a”, “b”, “e”, and “f” are as defined in claim 1, X¹ represents W or N, and X³ represents I or L;
SEQ ID NO: 49 (T) a (W)_{b}DNEG (X³) (F): (Y) f, wherein “a”, “b”, “e”, and “f” are as defined in claim 1, and X³ represents I or L, particularly L.

In another embodiment of the first aspect of the invention, optionally in combination with any of the embodiments provided above or below, the lipolytic peptide is a peptide of sequence SEQ ID NO: 1 or a salt thereof, made of L- and/or D-amino acids, as defined above, which is represented by SEQ ID NO. 73 and any salt thereof:
SEQ ID NO: 73 NDPEG (L)_{c}Y, wherein “c” is as defined above for SEQ ID NO. 1, and in claim 1.

In one embodiment of the first aspect of the invention, optionally in combination with any of the embodiments provided above or below, the lipolytic peptide is a peptide of sequence SEQ ID NO: 1 or a salt thereof, as defined above, which is selected from the group consisting of sequences SEQ ID NO: 47 to SEQ ID NO: 49 and SEQ ID NO: 73, wherein the sequence has one or more D-amino acids. In one embodiment of the first aspect of the invention, optionally in combination with any of the embodiments provided above or below, the lipolytic peptide is a peptide of sequence SEQ ID NO: 1 or a salt thereof, as defined above, which is selected from the group consisting of sequences SEQ ID NO: 50 to SEQ ID NO: 52 and SEQ ID NO:77. As well understood by a skilled person, SEQ ID NO: 50-52 have the same amino acid sequence as SEQ ID NO:47-49, respectively, but wherein each amino acid in each sequence is a D-amino acid.

In one embodiment of the first aspect of the invention, optionally in combination with any of the embodiments provided above or below, the lipolytic peptide is a peptide of sequence SEQ ID NO: 54 or a salt thereof, as defined above, wherein X¹ represents N; particularly X¹ represents the D-amino acid D-N.

In another embodiment of the first aspect of the invention, optionally in combination with any of the embodiments provided above or below, the lipolytic peptide is a peptide of sequence SEQ ID NO: 54 or a salt thereof, as defined above, wherein X² represents P, L, V, or A, particularly X² is a D-amino acid and represents D-P, D-L, D-V, or D-A.

In one embodiment of the first aspect of the invention, optionally in combination with any of the embodiments provided above or below, the lipolytic peptide is a peptide of sequence SEQ ID NO: 54 or a salt thereof, as defined above, wherein X³ and X⁴ are the same or different and represent V, L or A, particularly X³ and X⁴ are D-amino acids, the same or different, and represent D-V, D-L or D-A. In another particular embodiment of the first aspect of the invention, optionally in combination with any of the embodiments provided above or below, the lipolytic peptide is a peptide of sequence SEQ ID NO: 54 or a salt thereof, as defined above, wherein X³ represents V, L or A, particularly X³ is a D-amino acid and represents D-V, D-L or D-A. In another particular embodiment of the first aspect of the invention, optionally in combination with any of the embodiments provided above or below, the lipolytic peptide is a peptide of sequence SEQ ID NO: 54 or a salt thereof, as defined above, wherein X⁴ represents V, L or A, particularly X⁴ is a D-amino acid and represents D-V, D-L or D-A.

In another embodiment of the first aspect of the invention, optionally in combination with any of the embodiments provided above or below, the lipolytic peptide is a peptide of sequence SEQ ID NO: 54 or a salt thereof, as defined above, wherein X⁵ represents P or F, particularly X⁵ is a D-amino acid and represents D-P or D-F.

In one embodiment of the first aspect of the invention, optionally in combination with any of the embodiments provided above or below, the lipolytic peptide is a peptide of sequence SEQ ID NO: 54 or a salt thereof, made of L- and/or D-amino acids, as defined above, which is selected from the group consisting of sequences SEQ ID NO: 72, 73, 74 and 75 and any salt thereof, preferably from the group consisting of sequences SEQ ID NO: 72, 74 and 75 and any salt thereof:
SEQ ID NO:72 ND (X²)_{g}EG(X³)_{c} (X⁵)ₑY, wherein “g”, “c” and “e” are as defined above for SEQ ID NO:54 and in claim 1, X² represents V, A, L, or P, X³ represents L, V, or A, and X⁵ represents P or F.
SEQ ID NO: 73 NDPEG (L)_{c}Y, wherein “c” is as defined above for SEQ ID NO.54 or 1, and in claim 1.
SEQ ID NO:74 ND (X²) g (X³). (X⁵) Y, wherein “c”, “e” and “g” is as defined above for SEQ ID NO: 54 and in claim 1, X² represents V, A, or L; X³ represents L, V, A, and X⁵ represents F or P.
SEQ ID NO:75 ND (X²)_{g}EGLY, wherein “g” is as defined above for SEQ ID NO.54 and in claim 1, and X² is P or L.

In one embodiment of the first aspect of the invention, optionally in combination with any of the embodiments provided above or below, the lipolytic peptide is a peptide of sequence SEQ ID NO: 54 or a salt thereof, as defined above, which is selected from the group consisting of sequences SEQ ID NO: 72 to SEQ ID NO: 75, preferably from SEQ ID NO: 72, 74 and 75, wherein the sequence has one or more D-amino acids. In one embodiment of the first aspect of the invention, optionally in combination with any of the embodiments provided above or below, the lipolytic peptide is a peptide of sequence SEQ ID NO: 54 or a salt thereof, as defined above, which is selected from the group consisting of sequences SEQ ID NO: 76 to SEQ ID NO: 79, preferably from SEQ ID NO: 76, 78 and 79. As well understood by a skilled person, SEQ ID NO: 76-79 have the same amino acid sequence as SEQ ID NO:72-75, respectively, but wherein each amino acid in each sequence is a D-amino acid.

In one embodiment of the first aspect of the invention, optionally in combination with any of the embodiments provided above or below, the N-terminal end of the lipolytic peptide of the invention corresponds to —NH₂ or —NHC(O) R₃, being R₃ a (C₁-C₂₀)alkyl, particularly (C₁-C₁₅)alkyl, more particularly (C₁-C₁₀)alkyl. In a particular embodiment, the N-terminal end of the lipolytic peptide of the invention corresponds to —NH₂. In a particular embodiment, the N-terminal end of the lipolytic peptide of the invention corresponds to —NHC(O) R₃, being R₃ a (C₁-C₂₀)alkyl, preferably being R₃ a (C₁-C₁₅)alkyl, more preferably being R₃ a (C₁-C₁₀)alkyl.

In another particular embodiment, the N-terminal end of the lipolytic peptide of the invention corresponds to —NHC(O) R₃ and R₃ is —CH₃. In another particular embodiment, the N-terminal end of the lipolytic peptide of the invention corresponds to —NHC(O) R₃ and R₃ is CH₃CH₂. In another particular embodiment, the N-terminal end of the lipolytic peptide of the invention corresponds to —NHC(O) R₃ and R₃ is CH₃ (CH₂) 14.

In one embodiment of the first aspect of the invention, optionally in combination with any of the embodiments provided above or below, the C-terminal end of the lipolytic peptide is —COOH, —C(O) NH₂ or —C(O) NHR₆, wherein R₆ is (C₁-C₂₀)alkyl. In one embodiment of the first aspect of the invention, optionally in combination with any of the embodiments provided above or below, the C-terminal end of the lipolytic peptide is —COOH or —C(O) NH₂.

In one embodiment of the first aspect of the invention, optionally in combination with any of the embodiments provided above or below, the N-terminal end of the lipolytic peptide of the invention corresponds to —NH₂ or —NHC(O) R₃, being R₃ a (C₁-C₁₀)alkyl; and the C-terminal end of the lipolytic peptide is —COOH or —C(O) NH₂.

In one embodiment of the first aspect of the invention, optionally in combination with any of the embodiments provided above or below, the N-terminal end of the lipolytic peptide of the invention corresponds to —NH₂; and the C-terminal end of the lipolytic peptide is —COOH, —C(O) NH₂ or —C(O) NHR₆, wherein R₆ is (C₁-C₂₀)alkyl, preferably wherein the C-terminal end of the lipolytic peptide is —COOH or —C(O) NH₂.

In one embodiment of the first aspect of the invention, optionally in combination with any of the embodiments provided above or below, the N-terminal end of the lipolytic peptide of the invention corresponds to —NHC(O) R₃, being R₃ a (C₁-C₂₀)alkyl, preferably being R₃ a (C₁-C₁₅)alkyl, more preferably being R₃ a (C₁-C₁₀)alkyl; and the C-terminal end of the lipolytic peptide is —COOH, —C(O) NH₂ or —C(O) NHR₆, wherein R₆ is (C₁-C₂₀)alkyl, preferably wherein the C-terminal end of the lipolytic peptide is —COOH or —C(O) NH₂.

In one embodiment of the first aspect of the invention, optionally in combination with any of the embodiments provided above or below, the N-terminal end of the lipolytic peptide of the invention corresponds to —NHC(O) R₃ and R₃ is —CH₃; and the C-terminal end of the lipolytic peptide is —COOH, —C(O) NH₂ or —C(O) NHR₆, wherein R₆ is (C₁-C₂₀)alkyl, preferably wherein the C-terminal end of the lipolytic peptide is —COOH or —C(O) NH₂.

In one embodiment of the first aspect of the invention, optionally in combination with any of the embodiments provided above or below, the N-terminal end of the lipolytic peptide of the invention corresponds to —NHC(O) R₃ and R₃ is CH₃CH₂; and the C-terminal end of the lipolytic peptide is —COOH, —C(O) NH₂ or —C(O) NHR₆, wherein R₆ is (C₁-C₂₀)alkyl, preferably wherein the C-terminal end of the lipolytic peptide is —COOH or —C(O) NH₂.

In one embodiment of the first aspect of the invention, optionally in combination with any of the embodiments provided above or below, the N-terminal end of the lipolytic peptide of the invention corresponds to —NHC(O) R₃, being R₃ CH₃ (CH₂) 14; and the C-terminal end of the lipolytic peptide is —COOH, —C(O) NH₂ or —C(O) NHR₆, wherein R₆ is (C₁-C₂₀)alkyl, preferably wherein the C-terminal end of the lipolytic peptide is —COOH or —C(O) NH₂.

In another embodiment of the first aspect of the invention the peptide or salt thereof is selected from the group consisting of: SEQ ID NO: 3-38, 55-71, 80-87 and any salt thereof.

In another embodiment of the first aspect of the invention the peptide or salt thereof is selected from the group consisting of: SEQ ID NO: 3-38, 61, 66, 69, 80-87 and any salt thereof.

In another embodiment of the first aspect of the invention the peptide or salt thereof is selected from the group consisting of: SEQ ID NO: 55-60, 62-65, 67, 68, 70 and 71 and any salt thereof.

In a second and third aspect the present invention provides cosmetic and pharmaceutical compositions, respectively.

The compositions of the invention can also include one or more further lipolytic agent(s).

A “lipolytic agent”, in the context of the present invention, is understood as a compound that induces lipolysis, which is the enzymatic decomposition of triglycerides into glycerol and fatty acids. Illustrative non-limitative examples of further lipolytic agents are: phosphodiesterase inhibitors (e.g., xanthine derivatives), alpha-2 blockers compounds capable of blocking alpha-2 receptors at the adipocytes surface, beta-adrenergical agonists and antagonists (e.g. alverine and its organic or inorganic salts such as alverine citrate), agents inhibiting LDL and VLDL receptors synthesis, inhibitors of enzymes of fatty acid synthesis such as acetylCoA carboxylase, or fatty acid synthase or cerulenine, compounds stimulating beta receptors and/or G proteins, glucose transport blockers such as serutine or rutine, neuropeptide Y (NPY) antagonists capable of blocking NPY receptors at the adipocytes surface, cAMP and its cosmetically acceptable derivatives, adenylate cyclase enzyme active agents such as forskolin, agents modifying fat acids transport, lipolytic peptides and lipolytic proteins, like peptides or proteins such as the peptides derived from the parathyroidal hormone.

Other examples of usable lipolytic agents include botanical and marine extracts. Among plant extracts, there may more particularly be mentioned the extract of English ivy (Hedera Helix), of Chinese thorowax (Bupleurum chinensis), of arnica (Arnica Montana L), of rosemary (Rosmarinus officinalis N), of marigold (Calendula officinalis), of sage (Salvia officinalis L), of ginseng (Panax ginseng), of ginko biloba, of St.-John's-W ort (Hyperycum Perforatum), of butcher's-broom (Ruscus aculeatus L), of European meadowsweet (Filipendula ulmaria L), of big-flowered Jarva tea (Orthosiphon Stamincus Benth), of algae (Fucus Vesiculosus), of birch (Betula alba), of green tea, of cola nuts (Cola Nipida), of horse-chestnut, of bamboo, of spadeleaf (Centella asiatica), of heather, of fucus, of willow, of mouse-ear, extracts of escine, extracts of cangzhu, extracts of chrysanthellum indicum, extracts of the plants of the Armeniacea genus, Atractylodis Platicodon, Sinnomenum, Pharbitidis, Flemingia, extracts of Coleus such as C. Forskohlii, C. blumei, C. esquirolii, C. scutellaroides, C. xanthantus and C. Barbatus, such as the extract of root of Coleus barbatus, extracts of Ballote, extracts of Guioa, of Davallia, of Terminalia, of Barringtonia, of Trema, of antirobia, cecropia, argania, dioscoreae such as Dioscorea opposita or Mexican, as extracted of marine origin: extracts of algae or phytoplankton such as an extract of Laminaria digitata, diatoms, rhodysterol. All these extracts can of course to be taken in mixtures.

The compositions according to the invention can also contain in addition one or more additional active selected among: agents acting on the microcirculation (vasculoprotectors or vasodilators) such as the natural flavonoides, ruscogenines, esculosides, escine, nicotinates, heperidine methyl chalcone, butcher's-broom, essential oils of lavender or rosemary, the extracts of Ammi visnaga; anti-glycation agents such as extracts of Centella asiatica and Siegesbeckia, silicium, amadorine, ergothioneine and its derivatives, hydroxystilbenes and their derivatives (e.g. resveratrol), vegetable extracts of the family of Ericaceae, in particular bilberry extracts (Vaccinium angustifollium), vitamin C and its derivatives, retinol and its derivatives; as well as sunscreens of any nature.

The compositions of the invention may include various other and additional ingredients, which may be active, functional, conventionally used in cosmetic, personal care or topical/transdermal products or otherwise. Of course, a decision to include an additional ingredient and the choice of specific additional ingredients depends on the specific application and product formulation.

Thus, the compositions of the invention may include at least one skin care active. As used herein, “skin care actives” are additional ingredients, which provide some benefit to the object of the composition. Such additional ingredients may include one or more substances such as, without limitations, cleaning agents, hair conditioning agents, skin conditioning agents, hair styling agents, antidandruff agents, hair growth promoters, perfumes, sunscreen and/or sunblock compounds, pigments, moisturizers, film formers, hair colors, make-up agents, detergents, thickening agents, emulsifiers, humectants, emollients, antiseptic agents, deodorant actives, dermatologically acceptable carriers and surfactants.

The term “cosmetic composition” in accordance with the present invention relates to a formulation that can be used for cosmetic purposes, purposes as a basis for delivery of one or more cosmetic ingredients.

The term “cosmetically acceptable” refers to excipients or carriers that are appropriate for use in contact with human skin without inappropriate allergic response, instability, incompatibility, or toxicity among others.

The cosmetic composition encompasses the use of a cosmetically effective amount of the peptide(s) of the invention in mammals which are usually lower than the therapeutically effective amounts employed when SHBG is used as a medicament.

The term “SHBG” or “sex hormone binding globulin” as used herein refers to a protein encoded by the gene with Gene ID 6462 from NCBI (version 6 Nov. 2022).

In one embodiment, the cosmetic composition is a topical composition.

To increase the capability of the peptides to diffuse the skin, and thus being more active topically, it is advantageous to make them more lipophilic by grafting a fatty acid of more or less long chain (lipoyl, mirystyl, palmityl, stearyl, etc.) onto the N-terminal residue and/or esterifying the carboxyl group of the peptide, using well-routine protocols to those skilled in the art.

The topical cosmetic composition comprises appropriate cosmetic excipients or carriers for topical administration, including, but not limited to, repairing cutaneous barrier function agent, a hydrating agent, an emollient, an emulsifier, a thickener, a humectant, a pH-regulating agent, an antioxidant, a preservative agent, a vehicle, or their mixtures. The excipients or carriers used have affinity for the skin, are well tolerated, stable, and are used in an amount adequate to provide the desired consistency, and ease application.

Examples of appropriate topical skin barrier recovery agent include, but are not limited to, ceramides, cholesterol, fatty acids, and precursors of these lipids including cerebrosides, sphingoid bases such as phytosphingosine or sphingosine, or phospholipids including phosphatidylcholine, and agents that promote the synthesis of epidermal lipids like urea, dexpanthenol, and alpha-hydroxyacids including lactic acid among others.

Examples of appropriate topical hydrating agent include, but are not limited to, collagen, collagen amino acids, dimethiconol, glycine, hyaluronic acid, dimethylsilanol hyaluronate, magnesium stearate, maltitol, maltose, pyrrolidone carboxylic acid (PCA), manganese PCA, sodium PCA, mannitol, trehalose, trilactin, glucose, glutamic acid, hydrolyzed caesalpinia spinosa gum, caesalpinia spinosa gum, prunus persica extract, prunus serotina extract, echinacea angustifolia extract, Echinacea purpurea extract, methyl gluceth, hydrolyzed wheat gluten, erythritol, aluminium stearoyl glutamate, copper acetylmethionate, or ditridecyl dimmer dilinoleate. Particularly the hydrating agent is selected from the group consisting of glucose, glycine, lysine, glutamic acid, hydrolyzed caesalpinia spinosa gum, caesalpinia spinosa gum, sodium PCA, and their mixtures.

Examples of appropriate topical emollient agents include, but are not limited to, octyl hydroxystearate, lanolin, caprylic/capric triglyceride, cetyl palmitate, octyldodecanol, cetyl alcohol, isopropyl isostearate, glyceryl dilaurate, isopropyl myristate, palm alcohol, dimethicone, squalane, plukenetia volubilis seed oil, butyrospermum parkii butter, sucrose cocoate, or their mixtures. Particularly the emollient is selected from the group consisting of dimethicone, squalane, plukenetia volubilis seed oil, butyrospermum parkii butter, caprylic/capric triglyceride, octyldodecanol, or their mixtures.

Examples of appropriate emulsifier include, but are not limited to, glyceryl trioleate, glyceryl oleate, acetylated sucrose distearate, sorbitan trioleate, polyoxyethylene monostearate, glycerol monooleate, sucrose distearate, polyethylene glycol monostearate, octyl phenoxypoly (ethyleneoxy) ethanol, deacylerin penta-isostearate, sorbitan sesquioleate, hydroxylated lanolin, lecithin, lanolin, triglyceryl diisostearate, polyoxyethylene oleyl ether, calcium stearoyl-2-lactylate, sodium lauroyl lactylate, sodium stearoyl lactylate, cetearyl glucoside, methyl glucoside sesquistearate, sorbitan monopalmitate, methoxy polyethylene glycol-22/dodecyl glycol copolymer, polyethylene glycol-45/dodecyl glycol copolymer, polyethylene glycol 400 distearate and glyceryl stearate, candelilla/jojoba/rice bran polyglyceryl-3 esters, cetyl phosphate, potassium cetyl phosphate, or their mixtures. Particularly, the emulsifier is selected from the group consisting of glyceryl oleate, lecithin, sodium lauroyl lactylate, sodium stearoyl lactylate, glyceryl stearate, candelilla/jojoba/rice bran polyglyceryl-3 esters, and their mixtures.

Examples of appropriate surfactant agents include, but are not limited to, non-ionic, ionic (either anionic or cationic) or zwitterionic (or amphoteric wherein the head of the surfactant contains two oppositely charged groups) surfactants. Examples of anionic surfactants include, but are not limited to, those based on sulfate, sulfonate or carboxylate anions such as perfluorooctanoate (PFOA or PFO), alkyl benzene sulfonate, soaps, fatty acid salts, or alkyl sulfate salts such as perfluorooctanesulfonate (PFOS), sodium dodecyl sulfate (SDS), ammonium lauryl sulfate, or sodium lauryl ether sulfate (SLES). Examples of cationic surfactants include, but are not limited to, those based on quaternary ammonium cations such as or alkyltrimethylammonium including cetyl trimethylammonium bromide (CTAB) a.k.a., or hexadecyl trimethyl ammonium bromide, cetylpyridinium chloride (CPC), polyethoxylated tallow amine (POEA), benzalkonium chloride (BAC), or benzethonium chloride (BZT). Examples of zwitterionic surfactants include, but are not limited to dodecyl betaine, cocamidopropyl betaine, or coco ampho glycinate. Examples of non-ionic surfactants include, but are not limited to, alkyl poly (ethylene oxide), alkylphenol poly (ethylene oxide), copolymers of poly(ethylene oxide), poly (propylene oxide) (commercially called Poloxamers or Poloxamines), alkyl polyglucosides including octyl glucoside and decyl maltoside, fatty alcohols including cetyl alcohol and oleyl alcohol, cocamide MEA, cocamide DEA, or polysorbates including tween 20, tween 80, or dodecyl dimethylamine oxide. Particularly, the surfactant is foaming and skin friendly, including polysorbate 20 or 40, coco glucoside, lauryl glucoside, decyl glucoside, lauryl sulfates such as ammonium, sodium, magnesium, MEA, triethylamine (TEA), or mipa lauryl sulfate, cocamidopropyl betain, or sodium alkyl sulfosuccinates.

Examples of appropriate topical humectants include, but are not limited to, glycerin, diglycerin, ethylhexylglycerin, glucose, honey, lactic acid, polyethylene glycol, propylene glycol, sorbitol, sucrose, or threalose. Particularly, the humectant is selected from the group consisting of glycerin, diglycerin, ethylhexylglycerin, and their mixtures.

Examples of appropriate topical pH-regulating agents include, but are not limited to, acetic acid, lactic acid, citric acid, ethanolamine, formic acid, oxalic acid, potassium hydroxide, sodium hydroxide, triethanolamine, or their mixtures. Particularly, the pH-regulating agent is selected from the group consisting of triethanolamine, sodium hydroxide, lactic acid, and citric acid.

Examples of appropriate antioxidants include, but are not limited to, free radical scavengers or reducing agents such as, acetyl cysteine, ascorbic acid, ascorbyl palmitate, butylated hydroxytoluene, green tea extract, caffeic acid, cysteine, tocopherol, ubiquinone, propyl gallate, butylated hydroxytoluene (BHT), and their mixtures. Particularly, the antioxidant agent is selected from the group consisting of ascorbyl palmitate, and tocopherol.

Examples of appropriate preservative agents include, but are not limited to, benzoic acid, butylparaben, ethylparaben, propylparaben, methylparaben, sorbic acid, potassium sorbate, sodium benzoate, phenoxyethanol, triclosan, or their mixtures. Particularly, the preservative agent is selected from the group consisting of potassium sorbate, sodium benzoate, and phenoxyethanol.

Examples of appropriate viscosity agents include, but are not limited to, cellulose or their derivatives such as hydroxypropyl methylcellulose, polyethylene glycol, microcrystalline cellulose, cetearyl alcohol, alginates, branched polysaccharides, fumed silica, xanthan gum, carbomer, and polyacrylates. Particularly, the viscosity agent is selected from the group consisting of microcrystalline cellulose, cetearyl alcohol, cellulose, xanthan gum, and carbomer.

The compositions mentioned above also include a vehicle. Examples of vehicles include, but are not limited to, water, propylene glycol, butylene glycol, ethanol, isopropanol, or silicones.

The topical compositions of the invention can be formulated in several forms that include, but are not limited to, solutions, aerosols and non-aerosol sprays, shaving creams, powders, mousses, lotions, gels, sticks, ointments, pastes, creams, shampoos, shower gel, body washes or face washes.

Topical compositions of the present invention can be prepared according to methods well known in the state of the art. The appropriate excipients and/or carriers, and their amounts, can readily be determined by those skilled in the art according to the type of formulation being prepared.

In one embodiment of the second aspect of the invention, the topical cosmetic composition is in the form of a patch. The patch can be adhesive or non-adhesive. In one embodiment of the second aspect of the invention, optionally in combination with any of the embodiments provided above or below, the patch comprises a matrix which is capable of adhering to skin and comprises at least one polymer and one or more peptides, or acceptable salt(s) thereof, as defined according to the first aspect of the invention or any of the above embodiments.

The peptides of the invention may also be adsorbed on solid organic polymers or solid mineral substrates such as for example and not limited to, talc, bentonite, silica, starch or maltodextrin among others.

The compositions containing the peptides of the invention can also be incorporated into fabrics, non-woven fabrics and medical devices that are in direct contact with skin, so that they release the peptides of the present invention either by biodegradation of the anchoring system to the fabric or non-woven fabric or medical device or by friction with the body, by body moisture, by the pH of the skin or by body temperature. Furthermore, fabrics and non-woven fabrics can be used for making garments that are in direct contact with the body. Illustrative non-limitative examples of fabrics, non-woven fabrics, garments and medical devices are bandages, gauzes, shirts, socks, stockings, girdles, gloves, diapers, sanitary towels, dressings, bedspreads, wipes, hydrogels, adhesive patches, non-adhesive patches, microelectric patches and/or facial masks.

In another embodiment, the cosmetic composition of the second aspect of the invention is in an injectable form, either intra-epidermal, intra-dermal, and/or subcutaneous injectable form. In this embodiment, the composition comprises an injection medium, such as one or more macromolecules of the extracellular matrix, such as hyaluronic acid, collagen, and/or cells, and/or extracted from adipose tissue or preparation containing and/or cosmetically acceptable excipients or carriers, as disclosed above. In a particular embodiment, the composition in an injectable form is a solution, a gel or a hydrogel.

The cosmetic compositions, preferably the compositions in an injectable form as defined above, are also well suited for mesotherapy. Mesotherapy is a non-surgical cosmetic treatment technique involving intra-epidermal, intra-dermal, and/or subcutaneous injection of the agent(s) (micronutrients, vitamins, mineral salts, etc). The compositions are administered in the form of small multiple droplets into the epidermis, dermo-epidermal junction, and/or the dermis. To that end, the formulations of the disclosure can be injected utilizing needles with a diameter of about 0.26 to about 0.4 mm and a length ranging from about 4 to about 14 mm. Alternatively, the needles can be 21 to 32 G and have a length of about 4 mm to about 70 mm. Particularly, the needle is a single-use needle. The needle can be combined with a syringe, catheter, and/or a pistol (for example, a hydropneumatic-compression pistol). The formulations can be administered once or over several sessions with the subject spaced apart by a few days, or weeks. For instance, the subject can be administered a formulation every 1, 2, 3, 4, 5, 6, 7, days or every 1, 2, 3, or 4, weeks. The administration can be on a monthly or bi-monthly basis. Further, the formulation can be administered every 3, 6, 9, or 12 months.

The cosmetic composition of the invention may therefore be suitable for injection intra-skin, or for mesotherapy, by use of a syringe, using one or more micro-needles, an implant or a biodegradable reservoir.

The term “bio-degradable reservoir” indicates a device comprising degradable molecules in a more or less long time, often a few weeks to several months or even years after introduction into the skin.

In a particular embodiment, the composition suitable for intra-skin or for mesotherapy is as defined in any of the embodiments defining the cosmetic composition in an injectable form.

The invention also encompasses the use of a cosmetically effective amount of the peptide as defined in the first aspect of the invention or of the composition as defined in the second aspect of the invention, for improving the bodily appearance of a mammal with subcutaneous fat herniated or accumulated within the fibrous connective tissue under the skin. This aspect can also be formulated as a cosmetic method for improving the bodily appearance of a mammal with subcutaneous fat herniated or accumulated within the fibrous connective tissue under the skin comprising administering a cosmetically effective amount of the peptide as defined in the first aspect of the invention or of the composition as defined in the second aspect of the invention, together with cosmetically excipients or carriers.

Subcutaneous fat is found just beneath the skin, as opposed to visceral fat, which is found in the peritoneal cavity. Subcutaneous fat can be measured using body fat calipers giving a rough estimate of total body adiposity. This fat aids in the process of homeostasis, by forming a layer of insulation to slow heat loss.

The fibrous connective tissue is the fraction containing fibroblasts cells and extracellular matrix components from the hypodermis (or connective tissue). The whole connective tissue also comprises adipose cells and macrophages. The hypodermis is used mainly for fat storage. Thus, in one particular embodiment the cosmetic use is for a mammal with cellulite.

In particular, the cosmetic effect of the peptides, compositions, kits and devices of the invention, derive from the lipolytic effect provided by them. Therefore, the topical or dermal administration of the peptides of the invention leads to the reduction of the subcutaneous fat herniated or accumulated within the fibrous connective tissue, or which is the same, reduces cellulite manifested topographically as skin dimpling and nodularity, often on the pelvic region (specifically the buttocks), lower limbs, and abdomen in females.

The cosmetic use of the peptides and cosmetic composition according to the invention is for the application in mammals, in particular in humans, which are healthy subjects that need not have any diseases. That is, they can be subjects without obesity, overweight or not suffering fatty liver. The cosmetic use is only for esthetical purposes aiming to improve the bodily appearance, and not to treat any disease. In general terms, the cosmetically effective amounts are different than therapeutically effective amounts, since in a cosmetic use the peptide has only to reach the fat accumulated or stored at subcutaneous level.

In a particular embodiment, the use in cosmetics (or the cosmetic use) of the peptides and cosmetic composition according to the invention is not a surgical cosmetic use, a use in a surgical cosmetic method, or a use that comprises a surgical step. In another embodiment, the use of the peptide or of the cosmetic composition of the invention in improving the bodily appearance of a mammal with subcutaneous fat herniated or accumulated within the fibrous connective tissue under the skin is not a surgical use, a use in a surgical method, or a use that comprises a surgical step. In another embodiment, the cosmetic method of the invention is a non-surgical cosmetic method or a method that does not comprise a surgical step.

In a third aspect, the invention provides pharmaceutical compositions comprising the one or more peptides as defined in the first aspect of the invention and any of the above embodiments.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc., must also be “acceptable” in the sense of being compatible with the other ingredients of the formulation. Suitable carriers, excipients, etc. can be found in standard pharmaceutical texts, and include, as a way of example preservatives, agglutinants, humectants, emollients, and antioxidants.

The term “therapeutically effective amount” as used herein, means an amount of an active agent high enough to deliver the desired benefit (either the treatment or prevention of the illness), but low enough to avoid serious side effects within the scope of medical judgment.

In one embodiment of the third aspect of the invention, the pharmaceutical composition is an oral pharmaceutical composition. Illustrative non-limitative examples of oral pharmaceutical compositions are tablets, capsules, granules, or pellets.

The appropriate excipients and/or carriers, and their amounts, can readily be determined by those skilled in the art according to the type of formulation being prepared.

Due to their role as lipolytic agent, the peptides of the invention are also suitable for the treatment of a disease due to the accumulation of lipids in an internal organ such as liver, heart or kidney. Illustrative non-limitative examples of lipid accumulation disease are hepatic steatosis, diabetes or cardiovascular diseases. Accordingly, it is also part of the invention the peptides as defined in the first aspect of the invention and any of the embodiments provided above, for use in therapy, and, particularly for the treatment and/or prevention of a disease caused or associated to an accumulation of lipids in an internal organ.

This aspect of the invention can also be formulated as the use of a peptide as defined in the first aspect of the invention for the manufacture of a medicament for the treatment and/or prevention of a disease selected from the group consisting of overweight, obesity, hepatic steatosis, diabetes and cardiovascular diseases in mammals, including human.

The present invention also relates to a method of treatment and/or prevention of a disease selected from the group consisting of overweight, obesity, hepatic steatosis, diabetes and cardiovascular diseases in mammals, including human, the method comprising administering a therapeutically effective amount of the peptide as defined in the first aspect of the invention, together with pharmaceutically acceptable excipients and/or carriers.

In an embodiment of the fifth aspect of the invention, the use of the peptide as defined in the first aspect of the invention as lipolytic agent is not a therapeutic use or use in a therapeutic treatment. In another embodiment of the fifth aspect of the invention, the use of the peptide as defined in the first aspect of the invention as lipolytic agent is not a treatment by surgery, a use in a surgical treatment or a use that comprises a surgical step.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

### I. Synthesis of the Peptides of the Invention

Synthesis method A:

Fmoc-Solid Phase Synthesis Route for EDL3D: TWDPEGVIFY (All D amino acids)
1. Swelled the Fmoc-D-Tyr (tbu)-Resin in DCM for 30 mins
2. Washed with DMF three times; DBLK deprotection treatment for 15 mins; Washed with DMF six times
3. Coupled 0.26 g of Fmoc-D-Phe-OH, reacted for 30 mins
4. Tested qualified (solution was bright yellow, and resin transparent); repeated step 2
5. Coupled 0.24 g of Fmoc-allo-D-Ile-OH, 0.23 g of Fmoc-D-Val-OH, 0.20 g of Fmoc-D-Gly-OH, 0.29 g of Fmoc-D-Glu-OH, 0.23 g of Fmoc-D-Pro-OH, 0.28 g of Fmoc-D-Asp-OH, 0.36 g of Fmoc-D-Trp-OH sequentially, each reaction allowed 30 mins and followed with step 2
6. Coupled the last amino acid, 0.27 g of Fmoc-D-Thr (tbu)-OH, reacted for 30 mins, Tested qualified, Washed with DMF three times; DBLK deprotection treatment for 15 mins;
7. Washed with DMF. DCM and MeOH separately, each three times, then cleaved, and followed with purification and lyophilization.

### Synthesis Method B: Fmoc-Solid Phase Synthesis Route for EDL3D-A: Ac-TWDPEGVIFY (AII D Amino Acids)

1. Swelled the Fmoc-D-Tyr (tbu)-Resin in DCM for 30 mins
2. Washed with DMF three times; DBLK deprotection treatment for 15 mins; Washed with DMF six times
3. Coupled 0.26 g of Fmoc-D-Phe-OH, reacted for 30 mins
4. Tested qualified (solution was bright yellow, and resin transparent); repeated step 2
5. Coupled 0.24 g of Fmoc-allo-D-Ile-OH, 0.23 g of Fmoc-D-Val-OH, 0.20 g of Fmoc-D-Gly-OH, 0.29 g of Fmoc-D-Glu-OH, 0.23 g of Fmoc-D-Pro-OH, 0.28 g of Fmoc-D-Asp-OH, 0.36 g of Fmoc-D-Trp-OH sequentially, each reaction allowed 30 mins and followed with step 4 and 2
6. Coupled the last AA, 0.27 g of Fmoc-D-Thr (tbu)-OH, reacted for 30 mins, repeated step 4 and 2
7. Completed AC reaction with Acetic anhydride and 85% Azabenzene at certain ration, allowed 30 mins, repeated step 4
8. Washed with DMF, DCM and MeOH separately, each three times, then cleaved, and followed with purification and lyophilization.

### Synthesis Method C: Fmoc-Solid Phase Synthesis Route for EDL3D-NH₂: TWDPEGVIFY-NH3 (all D Amino Acids)

1. Swelled the Fmoc-Rink Amide MBHA Resin in DCM for 30 mins
2. Washed with DMF three times; DBLK deprotection treatment for 15 mins; Washed with DMF six times
3. Coupled 0.26 g of Fmoc-D-Tyr (tbu)-OH, reacted for 30 mins
4. Tested qualified (solution was bright yellow, and resin transparent); repeated step 2
5. Coupled 0.26 g of Fmoc-D-Phe-OH, 0.24 g of Fmoc-allo-D-Ile-OH, 0.23 g of Fmoc-D-Val-OH, 0.20 g of Fmoc-D-Gly-OH, 0.29 g of Fmoc-D-Glu-OH, 0.23 g of Fmoc-D-Pro-OH, 0.28 g of Fmoc-D-Asp-OH, 0.36 g of Fmoc-D-Trp-OH sequentially, each reaction allowed 30 mins and followed with step 4 and 2
6. Coupled the last AA, 0.27 g of Fmoc-D-Thr (tbu)-OH, reacted for 30 mins, repeated step 4 and Washed with DMF three times; DBLK deprotection treatment for 15 mins
7. Washed with DMF. DCM and MeOH separately, each three times, then cleaved, and followed with purification and lyophilization.

### Synthesis Method D: Fmoc-Solid Phase Synthesis Route for EDL3D-NH₂/Ac: Ac-TWDPEGVIFY-NH2 (all D Amino Acids)

1. Swelled the Fmoc-Rink Amide MBHA Resin in DCM for 30 mins
2 Washed with DMF three times; DBLK deprotection treatment for 15 mins; Washed with DMF six times
3. Coupled 0.26 g of Fmoc-D-Tyr (tbu)-OH, reacted for 30 mins
4. Tested qualified (solution was bright yellow, and resin transparent); repeated step 2
5. Coupled 0.26 g of Fmoc-D-Phe-OH, 0.24 g of Fmoc-allo-D-Ile-OH, 0.23 g of Fmoc-D-Val-OH, 0.20 g of Fmoc-D-Gly-OH, 0.29 g of Fmoc-D-Glu-OH, 0.23 g of Fmoc-D-Pro-OH, 0.28 g of Fmoc-D-Asp-OH, 0.36 g of Fmoc-D-Trp-OH sequentially, each reaction allowed 30 mins and followed with step 4 and 2
6. Coupled the last AA, 0.27 g of Fmoc-D-Thr (tbu)-OH, reacted for 30 mins, repeated step 4 and 2
7. Completed AC reaction with Acetic anhydride and 85% Azabenzene at certain ration, allowed 30 mins, repeated step 4
8. Washed with DMF. DCM and MeOH separately, each three times, then cleaved, and followed with purification and lyophilization.

### Synthesis Method E: Fmoc-Solid Phase Synthesis Route for GP3DM-25: Ac-NDLEGLY (all D Amino Acids)

1. Swelled the Fmoc-D-Tyr (tbu)-Wang Resin in DCM for 30 mins
2. Washed with DMF three times; DBLK deprotection treatment for 15 mins; Washed with DMF six times
3. Coupled 0.3 g of Fmoc-D-Leu-OH, reacted for 30 mins
4. Tested qualified (solution was bright yellow, and resin transparent); repeated step 2
5. Coupled 0.26 g of Fmoc-Gly-OH, 0.38 g of Fmoc-D-Glu (Otbu)-OH, 0.3 g of Fmoc-D-Leu-OH, 0.37 g of Fmoc-D-Asp (Otbu)-OH, 0.53 g of Fmoc-D-Asn (Trt)-OH sequentially, each reaction allowed 30 mins and followed with step 4 and 2
6. Coupled the last AA, 2.25 pyridine: 4.5 acetic anhydride ml of CH₃CO, reacted for 30 mins, repeated step 4 and Washed with DMF three times; DBLK deprotection treatment for 15 mins
7. Washed with DMF. DCM and MeOH separately, each three times, then cleaved, and followed with purification and lyophilization.

### Synthesis Method F: Fmoc-Solid Phase Synthesis Route for GP3DM-26: EtCONH-NDLEGLY (all D Amino Acids)

1. Swelled the Fmoc-D-Tyr (tbu)-Wang Resin in DCM for 30 mins
2. Washed with DMF three times; DBLK deprotection treatment for 15 mins; Washed with DMF six times
3. Coupled 0.3 g of Fmoc-D-Leu-OH, reacted for 30 mins
4. Tested qualified (solution was bright yellow, and resin transparent); repeated step 2
5. Coupled 0.26 g of Fmoc-Gly-OH, 0.38 g of Fmoc-D-Glu (Otbu)-OH, 0.3 g of Fmoc-D-Leu-OH, 0.37 g of Fmoc-D-Asp (Otbu)-OH, 0.53 g of Fmoc-D-Asn (Trt)-OH sequentially, each reaction allowed 30 mins and followed with step 4 and 2
6. Coupled the last AA, 0.066 g of CH₃CH₂CO, reacted for 30 mins, repeated step 4 and Washed with DMF three times; DBLK deprotection treatment for 15 mins
7. Washed with DMF, DCM and MeOH separately, each three times, then cleaved, and followed with purification and lyophilization.

### Synthesis Method G: Fmoc-Solid Phase Synthesis Route for GP3DM-27: Ac-NDPEGLY-NH2 (all D-Amino Acids)

1. Swelled the Fmoc-Rink Amide MBHA Resin in DCM for 30 mins
2. Washed with DMF three times; DBLK deprotection treatment for 15 mins; Washed with DMF six times
3. Coupled 0.41 g of Fmoc-D-Tyr (tBu)-OH, reacted for 30 mins
4. Tested qualified (solution was bright yellow, and resin transparent); repeated step 2
5. Coupled 0.3 g of Fmoc-D-Leu-OH, 0.26 g of Fmoc-Gly-OH, 0.38 g of Fmoc-D-Glu (Otbu)-OH, 0.3 g of Fmoc-D-Pro-OH, 0.37 g of Fmoc-D-Asp (Otbu)-OH, 0.53 g of Fmoc-D-Asn (Trt)-OH sequentially, each reaction allowed 30 mins and followed with step 4 and 2
6. Coupled the last AA, 2.25 pyridine: 4.5 acetic anhydride ml of CH₃CO, reacted for 30 mins, repeated step 4 and Washed with DMF three times; DBLK deprotection treatment for 15 mins
7. Washed with DMF. DCM and MeOH separately, each three times, then cleaved, and followed with purification and lyophilization.

### Synthesis Method H: Fmoc-Solid Phase Synthesis Route for GP3DM-28: CH₃ (CH₂) 14CONH-NDLEGLY (all D-Amino Acids)

1. Swelled the Fmoc-D-Tyr (tBu)-Wang Resin in DCM for 30 mins
2. Washed with DMF three times; DBLK deprotection treatment for 15 mins; Washed with DMF six times
3. Coupled 0.3 g of Fmoc-D-Leu-OH, reacted for 30 mins
4. Tested qualified (solution was bright yellow, and resin transparent); repeated step 2
5. Coupled 0.26 g of Fmoc-Gly-OH, 0.38 g of Fmoc-D-Glu (Otbu)-OH, 0.3 g of Fmoc-D-Leu-OH, 0.37 g of Fmoc-D-Asp (Otbu)-OH, 0.53 g of Fmoc-D-Asn (Trt)-OH sequentially, each reaction allowed 30 mins and followed with step 4 and 2
6. Coupled the last AA, 0.23 g of CH₃ (CH₂) 14CO, reacted for 30 mins, repeated step 4 and Washed with DMF three times; DBLK deprotection treatment for 15 mins
7. Washed with DMF. DCM and MeOH separately, each three times, then cleaved, and followed with purification and lyophilization.

### Synthesis Method I: Fmoc-Solid Phase Synthesis Route for GP3DM-29: Ac-NDLEGLY-NH2 (all D Amino Acids)

1. Swelled the Fmoc-Rink Amide MBHA Resin in DCM for 30 mins
2. Washed with DMF three times; DBLK deprotection treatment for 15 mins; Washed with DMF six times
3. Coupled 0.41 g of Fmoc-D-Tyr (tBu)-OH, reacted for 30 mins
4. Tested qualified (solution was bright yellow, and resin transparent); repeated step 2
5. Coupled 0.3 g of Fmoc-D-Leu-OH, 0.26 g of Fmoc-Gly-OH, 0.38 g of Fmoc-D-Glu (Otbu)-OH, 0.3 g of Fmoc-D-Leu-OH, 0.37 g of Fmoc-D-Asp (Otbu)-OH, 0.53 g of Fmoc-D-Asn (Trt)-OH sequentially, each reaction allowed 30 mins and followed with step 4 and 2
6. Coupled the last AA, 2.25 pyridine: 4.5 acetic anhydride ml of CH₃CO, reacted for 30 mins, repeated step 4 and Washed with DMF three times; DBLK deprotection treatment for 15 mins
7. Washed with DMF. DCM and MeOH separately, each three times, then cleaved, and followed with purification and lyophilization.

Purification method:

HPLC:
1: MS detection shows the product is correct
2: dissolve the peptide sample (0.5 mg), use HPLC (10-100% gradient) to get the real time of peak.

TABLE 1

| Column: | Kromasil C18, 4.6*250 mm, 5 μm | | |
|---|---|---|---|
| Solvent A | A: 0.1% Trifluoroacetic | | |
| | Acid in 100% Acetonitrile | | |
| Solvent B | B: 0.1% Trifluoroacetic | | |
| | Acid in 100% Water | | |
| | | A | B |
| Gradient: | 0.0 min | 10% | 90% |
| | 25.0 min | 100% | 0% |
| | 25.1 min | 100% | 0% |

| | |
|---|---|
| 30.0 min | Stop |

| | |
|---|---|
| Volume: | 20 μl |
| Wavelength | 220 nm |
| Flow rate: | 1.0 ml/min |

3: Make the gradient accordingly (ACN: H₂O=1:7 gradient=12-22%)

TABLE 2

| Column: | Hanbang DAC-HB100 | | |
|---|---|---|---|
| Solvent A | A: 0.1% Trifluoroacetic | | |
| | Acid in 100% Acetonitrile | | |
| Solvent B | B: 0.1% Trifluoroacetic | | |
| | Acid in 100% Water | | |
| | | A | B |
| Gradient: | 0.0 min | 12% | 88% |
| | 25.0 min | 22% | 78% |
| | 25.1 min | 100% | 0% |

| | |
|---|---|
| 30.0 min | Stop |

| | |
|---|---|
| Volume: | 300 ml |
| Wavelengt: | 220 nm |
| Flow rate: | 180.0 ml/min |

4. Purity detection: collect correct sample according to the detection

TABLE 3

| Column: | Kromasil C18, 4.6*250 mm, 5 μm | | |
|---|---|---|---|
| Solvent A | A: 0.1% Trifluoroacetic | | |
| | Acid in 100% Acetonitrile | | |
| Solvent B | B: 0.1% Trifluoroacetic | | |
| | Acid in 100% Water | | |
| | | A | B |
| Gradient: | 0.0 min | 15% | 85% |
| | 25.0 min | 40% | 60% |
| | 25.1 min | 100% | 0% |

| | |
|---|---|
| 30.0 min | Stop |

| | |
|---|---|
| Volume: | 20 μl |
| Wavelength: | 220 nm |
| Flow rate: | 1.0 ml/min |

5: rotary evaporate the collected correct product
6: lyophilization at −50° C.
7: Retest using HPLC to test the final purity of the dried product.

TABLE 4

| Column: | Kromasil C18, 4.6*250 mm, 5 μm | | |
|---|---|---|---|
| Solvent A | A: 0.1% Trifluoroacetic | | |
| | Acid in 100% Acetonitrile | | |
| Solvent B | B: 0.1% Trifluoroacetic% | | |
| | Acid in 100 Water | | |
| | | A | B |
| Gradient: | 0.0 min | 15% | 85% |
| | 25.0 min | 40% | 60% |
| | 25.1 min | 100% | 0% |

| | |
|---|---|
| 30.0 min | Stop |

| | |
|---|---|
| Volume: | 20 μl |
| Wavelength | 220 nm |
| Flow rate: | 1.0 ml/min |

MS:
1: take crude product 0.1 mg, dissolve it into ACN: H₂O=2:1 (looks transparent)
2: Wash the product using B-2060 sonic washer (30 seconds)
3: Centrifuge using RJ-TGL-16B centrifugal machine (1 min)
4. inject 0.5 ul into LCMS-2010EV MS detector to test the peak.

The characterization data of the peptides are provided in Table 5A and 5B below.

### II. Lipolytic Activity

Lipolytic activity was measured as glycerol (UM) release once either the caffeine or the peptides SEQ ID NO: 2 to 38 and 53 were added to a culture of differentiated 3T3-L1 cell line mouse adipocytes (Ref. JCRB9014-A) from Tebu-Bio (Tebu-bio, Barcelona, Spain). Briefly, mouse adipocytes (3T3-L1 cells) were cultured in 48 well plates as described previously (Saez-Lopez et al., 2020). Adipocytes were differentiated using cell culture reagents purchased from Tebu-Bio and following the manufacturer's instructions. At day of differentiation cells were treated in duplicates with vehicle (DMSO), Caffeine (100 μg/ml), and each of the peptides at the specified doses in Table 6a below. At the end of the experiment (12 h), supernatants were collected and glycerol content was measured using a Glycerol Assay Kit (Sigma) following manufacturer's instructions.

The same protocol was repeated again for some of the peptides, at a concentration of 100 μg/mL, but using human adipocytes (Tebu-Bio) instead of mice adipocytes. The results are summarized in Table 6b below. Lipolytic activity was measured as glycerol (UM) using a Lipolysis Assay kit from Tebu-bio (LIP-1—NC) following the manufacturer's instructions.

The results are summarized in Tables 6a and 6b below. As it can be derived, the peptides of the invention (SEQ ID NO: 3 to 38, 55-71, 80-87) were found to be remarkably more efficient than caffein after 12 hours. This is indicative of an improved efficiency which was found to be prolonged in time, at least for the 12 hours of the test, even using low doses.

On the other hand, Table 6b also supports the improved lipolytic effect provided by the peptides of the invention when compared to caffeine or Hexa-39.

### III. Time Course Lipolytic Activity

Lipolytic activity measured as glycerol (UM) release in differentiated human subcutaneous adipocytes treated for 120′ and 240′ with Caffeine at 10 μg/ml; or the peptide of sequence SEQ ID NO: 2, for comparative purpose, at 10 μg/ml; or the peptide of the invention of sequence SEQ ID NO: 3 at 10 μg/ml. At the end of the experiment, supernatants were collected and glycerol content was measured using a Lipolysis Assay kit from Tebu-bio (LIP-1—NC) following the manufacturer's instructions.

The results are summarized in Table 7 below. As it can be derived from the table below, the peptide of the invention provided a significant amount of glycerol at 120′, indicative of the lipolysis effect. On the contrary, neither caffeine nor the comparative peptide, at the same dose, provided any significant activity at that time.

On the other hand, Table 7 emphasizes again the improved efficiency in inducing lipolysis when the peptides of the invention are used, even after a very short period of time after the administration. In this case, peptide SEQ ID NO: 3 was found to be more than 24-fold more efficient at time 120′ vs caffeine or SEQ ID NO:2 and more than 5-fold at 240′.

In conclusion, the peptides of the invention not only are more efficient in providing a lipolytic activity, but also (1) induce the onset of the lipolytic activity remarkaly earlier, and (2) prolong the lipolytic effect for longer periods of time.

TABLE 5A

| SEQ | | | | | | | | | |
|---|---|---|---|---|---|---|---|---|---|
| ID | | Peptide | N- | C- | | Nr | Synth. | HPLC | |
| NO: | Product Name | seq. | term | Term | Modif. Seq | AA | method | Purity | MS |
| 2 | EDL 3L | TWDPEGVIFY | NH₂ | COOH | (I = L-Ile; All L aa) | 10 | A | 97.7% | 1226.8 |
| 3 | EDL 3D | TWDPEGVIFY | NH₂ | COOH | (I = D-Ile; All D aa) | 10 | A | 98.4% | 1224.6 |
| 4 | EDL 3D-Ac | TWDPEGVIFY | CH₃CONH | COOH | (All D amino acids) | 10 | B | 96.3% | 613.5 |
| 5 | EDL 3D-NH2 | TWDPEGVIFY | NH₂ | CONH₂ | (All D amino acids) | 10 | C | 96.6% | 633.0 |
| 6 | EDL 3D-NH2/Ac | TWDPEGVIFY | CH₃CONH | CONH₂ | (All D amino acids) | 10 | D | 96.2% | 632.6 |
| 7 | EDL 3D 9-I | WDPEGVIFY | NH₂ | COOH | (i = Alo-Ile, All D aa) | 9 | A | 97.6% | 1125.7 |
| 8 | EDL 3D 9-II | TDPEGVIFY | NH₂ | COOH | (i = Alo-Ile, All D aa) | 9 | A | 97.7% | 1040.7 |
| 9 | EDL 3D 9-III | TWPEGVIFY | NH₂ | COOH | (i = Alo-Ile, All D aa) | 9 | A | 98.1% | 1109.8 |
| 10 | EDL 3D 9-IV | TWDEGVIFY | NH₂ | COOH | (i = Alo-Ile, All D aa) | 9 | A | 95.4% | 1129.7 |
| 11 | EDL 3D 9-V | TWDPGVIFY | NH₂ | COOH | (i = Alo-Ile, All D aa) | 9 | A | 95.6% | 1097.7 |
| 12 | EDL P3D 9-VI | TWDPEVIFY | NH₂ | COOH | (i = Alo-Ile, All D aa) | 9 | A | 95.9% | 1169.8 |
| 13 | EDL 3D 9-VII | TWDPEGIFY | NH₂ | COOH | (i = Alo-Ile, All D aa) | 9 | A | 99.6% | 1127.7 |
| 14 | EDL 3D 9-VIII | TWDPEGVFY | NH₂ | COOH | (i = Alo-Ile, All D aa) | 9 | A | 99.2% | 1113.7 |
| 15 | EDL 3D 9-IX | TWDPEGVIY | NH₂ | COOH | (i = Alo-Ile, All D aa) | 9 | A | 95.2% | 1079.7 |
| 16 | EDL 3D 9-X | TWDPEGVIF | NH₂ | COOH | (i = Alo-Ile, All D aa) | 9 | A | 95.5% | 1063.7 |
| 17 | EDL 3D 8 I-II | DPEGVIFY | NH₂ | COOH | (i = Alo-Ile, All D aa) | 8 | A | 97.6% | 939.6 |
| 18 | EDL 3D 8 I-IX | WDPEGVIY | NH₂ | COOH | (i = Alo-Ile, All D aa) | 8 | A | 96.2% | 978.7 |
| 19 | EDL 3D 8 I-X | WDPEGVIF | NH₂ | COOH | (i = Alo-Ile, All D aa) | 8 | A | 95.9% | 962.7 |
| 20 | EDL 3D 8 II-IX | TDPEGVIY | NH₂ | COOH | (i = Alo-Ile, All D aa) | 8 | A | 96.0% | 893.6 |
| 21 | EDL 3D 8 II-X | TDPEGVIF | NH₂ | COOH | (i = Alo-Ile, All D aa) | 8 | A | 96.9% | 877.6 |
| 22 | EDL 3D 8 IX-X | TWDPEGVI | NH₂ | COOH | (i = Alo-Ile, All D aa) | 8 | A | 98.3% | 916.6 |
| 23 | EDL 3D 7 I-II- | DPEGVFY | NH₂ | COOH | (I = D-Ile; All D aa) | 7 | A | 97.0% | 826.6 |
| | VIII | | | | | | | | |
| 24 | EDL 3D 7 I-II- | DPEGVIY | NH₂ | COOH | (I = D-Ile; All D aa) | 7 | A | 98.0% | 792.6 |
| | IX | | | | | | | | |
| 25 | EDL 3D 7 I-II- | DPEGVIF | NH₂ | COOH | (I = D-Ile; All D aa) | 7 | A | 95.2% | 776.6 |
| | X | | | | | | | | |
| 26 | EDL 3D 7 I-IX- | WDPEGVI | NH₂ | COOH | (I = D-Ile; All D | 7 | A | 96.3% | 815.7 |
| | X | | | | amino acid) | | | | |
| 27 | EDL 3D 7 I- | WDPEGVY | NH₂ | COOH | (All D amino acid) | 7 | A | 95.4% | 865.6 |
| | VIII-IX | | | | | | | | |
| 28 | EDL 3D 7 I- | WDPEGVF | NH₂ | COOH | (All D amino acid) | 7 | A | 95.2% | 849.6 |
| | VIII-X | | | | | | | | |
| 29 | EDL 3D 6 I-II- | DPEGVI | NH₂ | COOH | (I = D-Ile; All D aa) | 6 | A | 98.0% | 629.4 |
| | IX-X | | | | | | | | |
| 30 | EDL 3D 6 I- | WDPEGV | NH₂ | COOH | (All D amino acid) | 6 | A | 97.8% | 702.5 |
| | VIII-IX-X | | | | | | | | |
| 31 | EDL 3D 6 VII- | TWDPEG | NH₂ | COOH | (All D amino acid) | 6 | A | 97.2% | 704.4 |
| | VIII-IX-X | | | | | | | | |
| 32 | EDL 3D 5 I- | WDPEG | NH₂ | COOH | (All D amino acid) | 5 | A | 96.3% | 603.4 |
| | VII-VIII-IX-X | | | | | | | | |
| 33 | EDL 3DM-1 | WDPEGLY | NH₂ | COOH | (All D amino acid) | 7 | A | 97.3% | 879.6 |
| 34 | EDL 3DM-2 | DPEGLFY | NH₂ | COOH | (All D amino acid) | 7 | A | 97.3% | 840.6 |
| 35 | EDL 3DM-3 | DPEGVLY | NH₂ | COOH | (All D amino acid) | 7 | A | 97.1% | 792.8 |
| 36 | EDL 3DM-4 | WDNEGLY | NH₂ | COOH | (All D amino acid) | 7 | A | 95.1% | 894.7 |
| 37 | EDL 3DM-5 | NDPEGLY | NH₂ | COOH | (All D amino acid) | 7 | A | 95.6% | 805.7 |
| 38 | EDL 3DM-6 | NDPEGVIY | NH₂ | COOH | (I = D-Ile; All D | 8 | A | 95.4% | 906.8 |
| | | | | | amino acid) | | | | |

TABLE 5B

| SEQ | | Peptide Sequence | | | | | | | |
|---|---|---|---|---|---|---|---|---|---|
| ID | Product | (D-aminoacids when | | | | Nr | Synth. | HPLC | |
| NO: | Name | not stated otherwise) | N-term | C-Term | Modif. Seq | AA | Method | Purity | MS |
| 55 | GP3DM-7 | NDVEGLY | NH₂ | COOH | (All D amino acid) | 7 | A | 95.5% | 808.85 |
| 56 | GP3DM-8 | NDAEGLY | NH₂ | COOH | (All D amino acid) | 7 | A | 95.5% | 780.80 |
| 57 | GP3DM-9 | NDLEGVY | NH₂ | COOH | (All D amino acid) | 7 | A | 95.7% | 808.85 |
| 58 | GP3DM-10 | NDLEGAY | NH₂ | COOH | (All D amino acid) | 7 | A | 96.1% | 780.80 |
| 59 | GP3DM-11 | NDLEGPY | NH₂ | COOH | (All D amino acid) | 7 | A | 98.7% | 806.83 |
| 80 | GP3DM-12 | LN-LD-LP-LE-LG-LL- | NH₂ | COOH | (All L amino acid) | 7 | A | 97.7% | 806.83 |
| | | LY | | | | | | | |
| 66 | Acetyl- | Ac-NDPEGLY | CH₃CONH | COOH | (All D amino acid) | 7 | B | 98.5% | 848.85 |
| | NH-EDL67 | | | | | | | | |
| 81 | Acetyl- | Ac-LN-LD-LP-LE-G- | CH₃CONH | COOH | (All L amino acid) | 7 | B | 97.1% | 848.87 |
| | NH-GP3DM- | LL-LY | | | | | | | |
| | 12 | | | | | | | | |
| 82 | GP3DM-13 | DN-LD-LP-LE-G-LL-LY | NH₂ | COOH | L,D-amino acid scan | 7 | A | 98.1% | 806.83 |
| 83 | GP3DM-14 | LN-DD-LP-LE-G-LL-LY | NH₂ | COOH | L,D-amino acid scan | 7 | A | 98.6% | 806.83 |
| 84 | GP3DM-15 | LN-LD-DP-LE-G-LL-LY | NH₂ | COOH | L,D-amino acid scan | 7 | A | 98.6% | 806.83 |
| 85 | GP3DM-16 | LN-LD-LP-DE-G-LL-LY | NH₂ | COOH | L,D-amino acid scan | 1 | A | 96.3% | 806.83 |
| 86 | GP3DM-17 | LN-LD-LP-LE-G-DL-LY | NH₂ | COOH | L,D-amino acid scan | 7 | A | 99.7% | 806.83 |
| 87 | GP3DM-18 | LN-LD-LP-LE-G-LL-DY | NH₂ | COOH | L,D-amino acid scan | 7 | A | 97.2% | 806.83 |
| 60 | GP3DM-19 | NDEGLFY | NH₂ | COOH | (All D amino acid) | 7 | A | 97.9% | 856.89 |
| 61 | GP3DM-20 | NDPEGY | NH₂ | COOH | (All D amino acid) | 6 | A | 98.3% | 693.67 |
| 62 | GP3DM-21 | NDLEGLY | NH₂ | COOH | (All D amino acid) | 7 | A | 96.4% | 822.90 |
| 63 | GP3DM-22 | NDEGLY | NH₂ | COOH | (All D amino acid) | 6 | A | 96.7% | 709.72 |
| 64 | GP3DM-23 | NDEGLY | NH₂ | COOH | (All D amino acid) | 6 | A | 96.3% | 692.73 |
| 65 | GP3DM-24 | NDEGLY | NH₂ | COOH | (All D amino acid) | 6 | A | 97.1% | 596.56 |
| 67 | GP3DM-25 | Ac-NDLEGLY | CH₃CONH | COOH | (All D amino acid) | 7 | E | 96.4% | 864.91 |
| 68 | GP3DM-26 | EtCONH-NDLEGLY | CH₃CH₂CONH | COOH | (All D amino acid) | 7 | F | 96.7% | 878.92 |
| 69 | GP3DM-27 | Ac-NDPEGLY-NH₂ | CH₃CONH | CONH₂ | (All D amino acid) | 7 | G | 95.4% | 847.89 |
| 70 | GP3DM-28 | CH₃(CH₂)₁₄CONH-NDLEGLY | CH₃(CH₂)₁₄CONH | COOH | (All D amino acid) | 7 | H | 96.4% | 1061.29 |
| 71 | GP3DM-29 | Ac-NDLEGLY-NH₂ | CH₃CONH | CONH₂ | (All D amino acid) | 7 | I | 99.2% | 863.93 |

TABLE 6a

| | | Lipolysis Activity measured as | | |
|---|---|---|---|---|
| | | Glycerol (μM) release in media of | | |
| | | 3T3-L1 mouse adipocytes (12 h) | | |
| SEQ ID NO: | Product Name | 1 μg/ml | 10 μg/ml | 100 μg/ml |
| — | Caffeine | | | 297 |
| 2 (comparative) | GP3L | 230 | 250 | 360 |
| 3 | GP3D | 538 | 574 | 580 |
| 4 | GP3D-Ac | 354 | 388 | 498 |
| 5 | GP3D-NH2 | 506 | 449 | 368 |
| 6 | GP3D-AcNH2 | 606 | 464 | 472 |
| 7 | GP3D-9 I | | | 344 |
| 8 | GP3D-9 II | | | 315 |
| 9 | GP3D-9 III | | | 346 |
| 11 | GP3D-9 V | | | 394 |
| 12 | GP3D-9 VI | | | 315 |
| 13 | GP3D-9 VII | | | 354 |
| 15 | GP3D-9 IX | | | 356 |
| 16 | GP3D-9 X | | | 493 |
| 17 | GP3D-8 I II | 401 | 474 | 468 |
| 18 | GP3D-8 I IX | 452 | 426 | 588 |
| 19 | GP3D-8 I X | 616 | 465 | 441 |
| 20 | GP3D-8 II IX | 557 | 508 | 370 |
| 21 | GP3D-8 II X | 391 | 434 | 493 |
| 22 | GP3D-8 IX X | 396 | 383 | 528 |
| 23 | GP3D-7 I | 485 | 325 | 340 |
| | II VIII | | | |
| 24 | GP3D-7 I | 417 | 380 | 346 |
| | II IX | | | |
| 25 | GP3D-7 I | 391 | 405 | 313 |
| | II X | | | |
| 27 | GP3D-7 I | — | 314 | — |
| | VIII IX | | | |
| 29 | GP3D-6 I | 326 | 300 | 368 |
| | II IX X | | | |
| 30 | GP3D-6 I | — | — | 360 |
| | VIII IX X | | | |
| 31 | GP3D-6 VII | 404 | 327 | — |
| | VIII IX X | | | |
| 32 | GP3D-5 I | 368 | 349 | 398 |
| | VII VIII IX X | | | |
| 33 | GP3D-DM1 | 356 | 477 | 324 |
| 34 | GP3D-DM2 | 303 | — | — |
| 35 | GP3D-DM3 | — | — | 307 |
| 36 | GP3D-DM4 | 469 | 416 | 309 |
| 37 | GP3D-DM5 | — | — | 326 |
| 38 | GP3D-DM6 | 304 | — | 405 |

TABLE 6b

| | Lipolysis Activity measured as Glycerol (μM) release | | | |
|---|---|---|---|---|
| | in media of human subcutaneoous mature adipocytes | | | |
| Product Name | 25 μg/ml | 50 μg/ml | 75 μg/ml | 100 μg/ml |
| Caffeine | | | | 35.53 |
| Hexa-39 | | | | 29.21 |
| GP3D-Ac | 16.56 | 36.41 | 43.36 | 58.41 |
| GP3D-7 I | 19.68 | 34.01 | 46.62 | 56.08 |
| II IX | | | | |
| GP3D-7 I | 17.04 | 34.80 | 48.09 | 58.31 |
| II X | | | | |
| GP3D-7 I | 14.57 | 36.68 | 42.95 | 46.85 |
| VIII X | | | | |
| GP3D-6 I | 17.01 | 40.79 | 43.36 | 53.13 |
| II IX X | | | | |
| GP3D-6 I | 18.07 | 32.53 | 47.82 | 51.90 |
| VIII IX X | | | | |
| GP3D-DM1 | 15.94 | 32.98 | 37.78 | 40.28 |
| GP3D-DM2 | 15.77 | 34.83 | 48.40 | 52.24 |
| GP3D-DM5 | 21.84 | 39.18 | 47.07 | 55.12 |
| GP3D-DM6 | 19.83 | 37.09 | 48.37 | 55.19 |
| Acetyl-NH- | 66.2 | 121.0 | | 74.2 |
| EDL67 | | | | |
| Acetyl-NH- | | | | 138.6 |
| GP3DM-12 | | | | |
| GP3DM-13 | | 49.5 | | 42.1 |
| GP3DM-14 | | 53.1 | | 52.8 |
| GP3DM-15 | | 42.0 | | 47.2 |
| GP3DM-16 | | 41.5 | | 44.1 |
| GP3DM-17 | | 47.4 | | 53.6 |
| GP3DM-18 | | 44.4 | | 63.5 |
| GP3DM-19 | 87.1 | 64.0 | | 123.3 |
| GP3DM-20 | 128.1 | 117.7 | | 118.7 |
| GP3DM-21 | 116.5 | 116.7 | | 102.5 |
| GP3DM-22 | 52.1 | 59.5 | | 131.9 |
| GP3DM-23 | 139.7 | 97.7 | | 170.0 |
| GP3DM-24 | 105.2 | 111.0 | | 148.6 |
| GP3DM-25 | 134.2 | 104.4 | | 121.6 |
| GP3DM-26 | 98.4 | 140.0 | | 149.6 |
| GP3DM-27 | 72.6 | 133.3 | | 142.7 |
| GP3DM-28 | 50.5 | 122.6 | | 106.0 |
| GP3DM-29 | 123.8 | 72.4 | | 131.9 |

TABLE 7

| Time | Caffeine | SEQ ID NO: 2 | SEQ ID NO: 3 |
|---|---|---|---|
| (min) | (10 ug/ml) | (10 ug/ml) | (10 ug/ml) |
| 120 | −3.0856 | −0.5343 | 24.8964 |
| 240 | 17.1602 | 17.7363 | 90.1603 |

### Clauses

For reasons of completeness, various aspects of the invention are set out in the following numbered clauses:

Clause 1. A lipolytic peptide which:
(a) has a sequence length from 5 to 9 amino acids, each amino acid being either L- or D-amino acid, the peptide corresponding to a sequence SEQ ID NO: 54 or an acceptable salt thereof:

(T)ₐ(X¹)_{b}D(X²)_{g}EG(X³)_{c}(X⁴)_{d}(X⁵)ₑ(Y)_{f}

wherein:
X¹ represents W, N, Q or K;
X² represents P, N, a basic amino acid or a hydrophobic amino acid
X³ and X⁴ are the same or different and represent A, V, L or I;
X⁵ represents F or P,
subindexes “a”, “b”, “c”, “d”, “e”, “f” and “g” are integer numbers independently selected from 0 and 1; provided that two, three, four, five or six of the subindexes “a”, “b”, “c”, “d”, “e”, “f” and “g” represent 1; and the remaining subindexes represent zero;
   or, alternatively,
(b) is selected from the group consisting of: SEQ ID NO: 39-46 and any salt thereof;
   and wherein
   the N-terminal end of the lipolytic peptide corresponds to —NR₁R₂, being R₁ and R₂ independently selected from the group consisting of —H, —C(O) R₃, a triglyceride and (C₁-C₂₀)alkyl;
   the C-terminal end of the lipolytic peptide corresponds to —COOR₄ or —C(O) NR₅R₆; and
   R₃ to R₆ are radicals independently selected from the group consisting of:—H and (C₁-C₂₀)alkyl.
   Clause 2. A lipolytic peptide which:
(a) has a sequence length from 5 to 9 amino acids, each one of the amino acids being a L- or D-amino acid, and corresponds to sequence SEQ ID NO: 1 or an acceptable salt thereof:

(T)ₐ(X¹)_{b}D(X²)EG(X³)_{c}(X⁴)_{d}(F)ₑ(Y)_{f}

wherein:
X¹ represents W, N, Q or K;
X² represents P, N or a basic amino acid;
X³ and X⁴ are the same or different and represent A, V, L or I;
subindexes “a”, “b”, “c”, “d”, “e”, and “f” are integer numbers independently selected from 0 and 1; provided that one, two, three, four or five of the subindexes “a”, “b”, “c”, “d”, “e”, and “f” represent 1; and the remaining subindexes represent zero;
   or, alternatively,
(b) is selected from the group consisting of: SEQ ID NO: 39-46 and any acceptable salt thereof;
   and wherein
   the N-terminal end of the lipolytic peptide corresponds to —NR₁R₂, being R₁ and R₂ independently selected from the group consisting of —H, —C(O) R₃, a triglyceride and (C₁-C₂₀)alkyl;
   the C-terminal end of the lipolytic peptide corresponds to —COOR₄ and —C(O) NR₅R₆; and
   R₃ to R₆ are radicals independently selected from the group consisting of:—H and (C₁-C₂₀)alkyl.
   Clause 3. A lipolytic peptide which:
(a) has a sequence length from 5 to 9 amino acids, each one of the amino acids being a L- or D-amino acid, and corresponds to sequence SEQ ID NO: 1 or an acceptable salt thereof:

(T)ₐ(X¹)_{b}D(X²)EG(X³)_{c}(X⁴)_{d}(F)ₑ(Y)_{f}

wherein:
X¹ represents W, N or Q;
X² represents P, N or a basic amino acid;
X³ and X⁴ are the same or different and represent A, V, L or I;
subindexes “a”, “b”, “c”, “d”, “e”, and “f” are integer numbers independently selected from 0 and 1; provided that one, two, three, four or five of the subindexes “a”, “b”, “c”, “d”, “e”, and “f” represent 1; and the remaining subindexes represent zero;
   or, alternatively,
(b) is selected from the group consisting of: SEQ ID NO: 39-46 and any acceptable salt thereof;
   and wherein
   the N-terminal end of the lipolytic peptide corresponds to —NR₁R₂, being R₁ and R₂ independently selected from the group consisting of —H, —C(O) R₃, a triglyceride and (C₁-C₂₀)alkyl;
   the C-terminal end of the lipolytic peptide corresponds to —COOR₄ and —C(O) NR₅R₆; and
   R₃ to R₆ are radicals independently selected from the group consisting of:—H and (C₁-C₂₀)alkyl.
   Clause 4. A lipolytic peptide which:
(a) has a sequence length from 5 to 9 amino acids, each one of the amino acids being a L- or D-amino acid, and corresponds to sequence SEQ ID NO: 1 or an acceptable salt thereof:

(T)ₐ(X¹)_{b}D(X²)EG(X³)_{c}(X⁴)_{d}(F)ₑ(Y)_{f}

wherein:
X¹ represents W or N;
X² represents P, N or a basic amino acid;
X³ and X⁴ are the same or different and represent A, V, L or I;
subindexes “a”, “b”, “c”, “d”, “e”, and “f” are integer numbers independently selected from 0 and 1; provided that one, two, three, four or five of the subindexes “a”, “b”, “c”, “d”, “e”, and “f” represent 1; and the remaining subindexes represent zero;
   or, alternatively,
(b) is selected from the group consisting of: SEQ ID NO: 39-46 and any acceptable salt thereof;
   and wherein
   the N-terminal end of the lipolytic peptide corresponds to —NR₁R₂, being R₁ and R₂ independently selected from the group consisting of —H, —C(O) R₃, a triglyceride and (C₁-C₂₀)alkyl;
   the C-terminal end of the lipolytic peptide corresponds to —COOR₄ and —C(O) NR₅R₆; and
   R₃ to R₆ are radicals independently selected from the group consisting of:—H and (C₁-C₂₀)alkyl.
   Clause 5. The lipolytic peptide of any one of the preceding clauses, which is a peptide selected from the sequences SEQ ID NO: 39-42 wherein two or more of the amino acids are D-amino acids, particularly all the amino acids are D-amino acids; or, alternatively, it is selected from sequences SEQ ID NO: 43-46 and any acceptable salt thereof, wherein one or more of the amino acids forming the peptide are D-amino acids, particularly all the amino acids forming the peptide are D-amino acids.
   Clause 6. The lipolytic peptide of any one of the preceding clauses, wherein X¹ represents W or N, preferably W; particularly X¹ is a D-amino acid D-W or D-N.
   Clause 7. The lipolytic peptide of any one of the preceding clauses, which is a peptide of sequence SEQ ID NO: 1 or a salt thereof, as defined above, wherein X² represents P or N; X² represents a D-amino acid selected from D-P and D-N.
   Clause 8. The lipolytic peptide of any one of the preceding clauses, which is a peptide of sequence SEQ ID NO: 1 or a salt thereof, as defined above, wherein X³ and X⁴ are the same or different and represent V, L or I; particularly, X³ and X⁴ are the same or different and represent D-amino acids, selected from D-V, D-L and D-I.
   Clause 9. The lipolytic peptide of any of the preceding clauses, which is selected from the group consisting of sequences SEQ ID NO: 47 to SEQ ID NO: 49 and any salt thereof:
SEQ ID NO: 47 (T) a (X¹)_{b}DPEGV (X⁴). (F) e (Y) f, wherein “a”, “b”, “d”, “e”, and “f” are as defined in clause 1, X¹ represents W or N, and X⁴ represents I or L;
SEQ ID NO: 48 (T) a (X¹)_{b}DPEGX³ (F): (Y) f, wherein “a”, “b”, “e”, and “f” are as defined in clause 1, X¹ represents W or N, and X³ represents I or L;
SEQ ID NO: 49 (T)ₐ (W)_{b}DNEG(X³)(F)ₑ(Y)_{f}, wherein “a”, “b”, “e”, and “f” are as defined in clause 1, and X³ represents I or L, particularly L.
   Clause 10. The lipolytic peptide of any of the preceding clauses, which is of sequence SEQ ID NO. 73 and any salt thereof:
SEQ ID NO: 73 NDPEG (L) cY, wherein “c” is as defined above for SEQ ID NO. 1, and in clause 1.
   Clause 11. The lipolytic peptide of any one of the preceding clauses, which is a peptide of sequence SEQ ID NO: 1, 43-48 or an acceptable salt thereof, and one or more of the amino acids forming the peptide are D-amino acid(s).
   Clause 12. The lipolytic peptide of any one of the preceding clauses, which is a peptide of sequence SEQ ID NO: 49 or 73 or an acceptable salt thereof, and one or more of the amino acids forming the peptide are D-amino acid(s).
   Clause 13. The peptide of any one of clauses 11 or 12, wherein all the amino acids forming the peptide are D-amino acids.
   Clause 14. The lipolytic peptide of clause 1, wherein the peptide with a sequence length from 5 to 9 amino acids corresponds to sequence SEQ ID NO:54, wherein X¹ represents N, particularly X¹ represents D-N.
   Clause 15. The lipolytic peptide of any one of clauses 1 or 14, wherein the peptide with a sequence length from 5 to 9 amino acids corresponds to sequence SEQ ID NO:54, wherein X² represents P, L, V, or A, particularly D-P, D-L, D-V, or D-A.
   Clause 16. The lipolytic peptide of any one of clauses 1, 14 or 15, wherein the peptide with a sequence length from 5 to 9 amino acids corresponds to sequence SEQ ID NO:54, wherein X³ and X⁴ are the same or different and represent L, V, or A; particularly X³ and X⁴ are the same or different and represent D-L, D-V, or D-A.
   Clause 17. The lipolytic peptide of any one of clauses 1, 14-16, wherein the peptide with a sequence length from 5 to 9 amino acids corresponds to sequence SEQ ID NO:54, wherein X⁵ represents F or P, particularly D-F or D-P.
   Clause 18. The lipolytic peptide of any one of clauses 1, 14-17, which is a peptide of sequence SEQ ID NO: 54, 72-75 or a salt thereof:
SEQ ID NO:72 ND (X²)_{g}JEG (X³)_{c}(X⁵)ₑY, wherein “g”, “c” and “e” are as defined above for SEQ ID NO:54 and in clause 1, X² represents V, A, L, or P, X³ represents L, V, or A, and X⁵ represents P or F.
SEQ ID NO: 73 NDPEG (L)_{c}Y, wherein “c” is as defined above for SEQ ID NO.54 or 1, and in clause 1.
SEQ ID NO:74 ND (X²) g (X³). (X⁵)ₑY, wherein “c”, “e” and “g” is as defined above for SEQ ID NO: 54 and in clause 1, X² represents V, A, or L; X³ represents L, V, A, and X⁵ represents F or P.
SEQ ID NO:75 ND (X²)_{g}EGLY, wherein “g” is as defined above for SEQ ID NO.54 and in clause 1, and X² is P or L.
   Clause 19. The lipolytic peptide of any one of clauses 1, 14-18, which is a peptide of sequence SEQ ID NO: 54, 72, 74 or 75 or a salt thereof.
   Clause 20. The lipolytic peptide of any one of clauses 1, 14-18, which is a peptide of sequence SEQ ID NO: 54, 72-75 or an acceptable salt thereof, and one or more of the amino acids forming the peptide are D-amino acid(s).
   Clause 21. The lipolytic peptide of any one of clauses 1, 14-19, which is a peptide of sequence SEQ ID NO: 54, 72, 74 or 75 or an acceptable salt thereof, and one or more of the amino acids forming the peptide are D-amino acid(s).
   Clause 22. The lipolytic peptide of any one of clauses 20 or 21 wherein all the amino acids forming the peptide are D-amino acids.
   Clause 23. The peptide or salt thereof according to any one of the preceding clauses, wherein the N-terminal end is —NH₂ or —NHC(O) R₃, being R₃ a (C₁-C₂₀)alkyl.
   Clause 24. The peptide or salt thereof according to any one of the preceding clauses, wherein the N-terminal end is —NHC(O) R₃, being R₃ a (C₁-C₁₅)alkyl.
   Clause 25. The peptide or salt thereof according to any one of the preceding clauses, wherein the N-terminal end is —NHC(O) R₃, being R₃ a (C₁-C₁₀)alkyl.
   Clause 26. The peptide or salt thereof according to any one of the preceding clauses, wherein the C-terminal end is —COOH, —C(O) NH₂, —C(O) NHR₆, wherein R₆ is (C₁-C₂₀)alkyl.
   Clause 27. The peptide or salt thereof according to any one of the preceding clauses, which is selected from the group consisting of: SEQ ID NO: 3-38, 55-71, 80-87 and any acceptable salt thereof.
   Clause 28. The peptide or salt thereof according to any one of the preceding clauses, which is selected from the group consisting of: SEQ ID NO: 3-38, 61, 66, 69, 80-87 and any acceptable salt thereof.
   Clause 29. The peptide or salt thereof according to any one of the preceding clauses, which is selected from the group consisting of: SEQ ID NO: 55-60, 62-65, 67, 68, 70 and 71 and any acceptable salt thereof.
   Clause 30. A cosmetic composition comprising a cosmetically effective amount of a peptide or salt thereof, as defined in any one of the preceding clauses, together with one or more cosmetically acceptable carriers or excipients.
   Clause 31. The cosmetic composition of clause 30, which further comprises one or more of further lipolytic agents, vasculoprotector or vasodilators, retinol, sunscreens, vitamins, alpha-hydroxy acids, surfactants, dyes, fragrances and pigments.
   Clause 32. The cosmetic composition of clause 30 or 31, which is a topical or an injectable cosmetic composition.
   Clause 33. The cosmetic composition of any of the clauses 30-32, which is a topical composition selected from solutions, aerosols and non-aerosol sprays, shaving creams, powders, mousses, lotions, gels, sticks, ointments, pastes, creams, shampoos, shower gel, body washes or face washes.
   Clause 34. The cosmetic composition of any one of the clauses 30-31, which is in the form of a patch.
   Clause 35. An injection device, such as an intra-dermal injection device, prefilled with the peptide as defined in any of the preceding clauses 1-29 or the cosmetic composition as defined in any of the preceding clauses 30-31.
   Clause 36. A pharmaceutical composition comprising a therapeutically effective amount of a peptide as defined in any of the clauses 1-29 and one or more therapeutically acceptable excipients or carriers.
   Clause 37. A kit of parts comprising
a peptide as defined in any one of the preceding clauses 1-29 or the cosmetic composition as defined in clause 30-31 or the pharmaceutical composition as defined in clause 36, and
a device selected from the group consisting of: an intra-dermal injection device, an implant, or a biodegradable container.
   Clause 38. Use of the peptide as defined in any of the preceding clauses 1-29 as lipid reducing agent, particularly as lipolytic agent.
   Clause 39. The use of the peptide as defined in clause 38, wherein said use is not a therapeutic use, a treatment by surgery or is not use comprising a step of treatment by surgery.
   Clause 40. Use of the peptide as defined in any one of the preceding clauses 1-29 or of the cosmetic composition as defined in any one of the clauses 30-34 or of the injection device of clause 35 or of the kit of clause 37 in cosmetics.
   Clause 41. The use according to clause 40, wherein said use is not a surgical cosmetic use, a use in a surgical cosmetic method or a use comprising a step of treatment by surgery.
   Clause 42. A peptide as defined in any one of the preceding clauses 1-29 or of the pharmaceutical composition as defined in clause 36 or of the kit of clause 37 for use in therapy.
   Clause 43. Use of a cosmetically effective amount of the peptide as defined in any one of the preceding clauses 1-29 or of the cosmetic composition as defined in any of the clauses 30-34 or of the injection device as defined in 35 or of the kit of parts as defined in 37 in improving the bodily appearance of a mammal with subcutaneous fat herniated or accumulated within the fibrous connective tissue under the skin.
   Clause 44. The use according to clause 43 wherein said use is not a surgical use, a use a in a surgical method or a use comprising a step of treatment by surgery.
   Clause 45. A peptide as defined in any one of the preceding clauses 1-29 or of the pharmaceutical composition as defined in clause 36 or of the kit of parts as defined in 37 for use in the treatment of the accumulation of lipids in internal organs.

### CITATION LIST

### Non Patent Literature

Saez-Lopez C. et al., “Sex hormone-binding globulin overexpression protects against high-fat diet-induced obesity in transgenic male mice”, J Nutr Biochem., 2020, 85:108480. doi: 10.1016/j.jnutbio.2020.108480

What is claimed is:

1. A lipolytic peptide which:
(a) has a sequence length from 5 to 9 amino acids, each amino acid being either L- or D-amino acid, the peptide corresponding to a sequence SEQ ID NO: 54 or an acceptable salt thereof:
(T)ₐ(X¹)_{b}D(X²)_{g}EG(X³)_{c}(X⁴)_{d}(X⁵)ₑ(Y)_{f}
wherein:
X¹ represents W, N, Q or K;
X² represents P, N, a basic amino acid or a hydrophobic amino acid
X³ and X⁴ are the same or different and represent A, V, L or I;
X⁵ represents F or P,
subindexes “a”, “b”, “c”, “d”, “e”, “f” and “g” are integer numbers independently selected from 0 and 1; provided that two, three, four, five or six of the subindexes “a”, “b”, “c”, “d”, “e”, “f” and “g” represent 1; and the remaining subindexes represent zero;
or, alternatively,
(b) is selected from the group consisting of: SEQ ID NO: 39-46 and any salt thereof;
and wherein
the N-terminal end of the lipolytic peptide corresponds to —NR₁R₂, being R₁ and R₂ independently selected from the group consisting of —H, —C(O) R₃, a triglyceride and (C₁-C₂₀)alkyl;
the C-terminal end of the lipolytic peptide corresponds to —COOR₄ or —C(O) NR₅R₆; and
R₃ to R₆ are radicals independently selected from the group consisting of:—H and (C₁-C₂₀)alkyl.

2. The lipolytic peptide of claim 1, wherein the peptide with a sequence length from 5 to 9 amino acids corresponds to sequence SEQ ID NO: 1 or a salt thereof:
(T)ₐ(X¹)_{b}D(X²)EG(X³)_{c}(X⁴)_{d}(F)ₑ(Y)_{f}
wherein:
X¹ represents W, N, Q or K;
X² represents P, N or a basic amino acid;
X³ and X⁴ are the same or different and represent A, V, L or I;
subindexes “a”, “b”, “c”, “d”, “e”, and “f” are integer numbers independently selected from 0 and 1; provided that one, two, three, four or five of the subindexes “a”, “b”, “c”, “d”, “e”, and “f” represent 1; and the remaining subindexes represent zero.

3. The lipolytic peptide of claim 2, which is a peptide of sequence SEQ ID NO: 1 having one or more of the following features:
X¹ represents W or N, particularly X¹ represents D-W or D-N;
X² represents P or N; particularly X² represents D-P or D-N; and
X³ and X⁴ are the same or different and represent V, L or I; particularly X³ and X⁴ are the same or different and represent D-V, D-L or D-I.

4. The lipolytic peptide of claim 1, which is a peptide of sequence SEQ ID NO: 1, 43-49, or 73 or a salt thereof, and one or more of the amino acids forming the peptide are D-amino acid(s); particularly all the amino acids forming the peptide are D-amino acids.

5. The lipolytic peptide of claim 1, wherein the peptide with a sequence length from 5 to 9 amino acids corresponds to sequence SEQ ID NO:54 having one or more of the following features:
X¹ represents N, particularly X¹ represents D-N;
X² represents P, L, V, or A, particularly D-P, D-L, D-V, or D-A,
X³ and X⁴ are the same or different and represent L, V, or A; particularly X³ and X⁴ are the same or different and represent D-L, D-V, or D-A,
X⁵ represents F or P, particularly D-F or D-P.

6. The lipolytic peptide of claim 1, which is a peptide of sequence SEQ ID NO: 54, 72, 74, 75 or a salt thereof, and one or more of the amino acids forming the peptide are D-amino acid(s); particularly all the amino acids forming the peptide are D-amino acids.

7. The peptide or salt thereof according to claim 1, wherein the N-terminal end is —NH₂ or —NHC(O) R₃, being R₃ a (C₁-C₂₀)alkyl, particularly a (C₁-C₁₀)alkyl; and the C-terminal end is —COOH or —C(O) NH₂.

8. The peptide or salt thereof according to claim 1 or, which is selected from the group consisting of: SEQ ID NO: 3-38, 61, 66, 69, 80-87 and any salt thereof.

9. The peptide or salt thereof according to claim 1, which is selected from the group consisting of SEQ ID NO: 55-60, 62-65, 67, 68, 70 and 71 and any salt thereof.

10. A cosmetic composition comprising a cosmetically effective amount of a peptide or salt thereof, as defined in claim 1, together with one or more cosmetically acceptable carriers or excipients.

11. The cosmetic composition of claim 10, which further comprises one or more of further lipolytic agents, vasculoprotector or vasodilators, retinol, sunscreens, vitamins, alpha-hydroxy acids, surfactants, dyes, fragrances and pigments.

12. The cosmetic composition of claim 10, which is a topical or an injectable cosmetic composition.

13. The cosmetic composition of claim 10, which is a topical composition selected from solutions, aerosols and non-aerosol sprays, shaving creams, powders, mousses, lotions, gels, sticks, ointments, pastes, creams, shampoos, shower gel, body washes or face washes.

14. The cosmetic composition of claim 10, which is an injectable cosmetic composition, preferably a subcutaneous injectable composition, selected from a solution, a gel or a hydrogel.

15. A pharmaceutical composition comprising a therapeutically effective amount of a peptide or salt thereof as defined in claim 1, together with one or more pharmaceutically acceptable carriers or excipients.

16-18. (canceled)

19. A cosmetic method for improving the bodily appearance of a mammal with subcutaneous fat herniated or accumulated within the fibrous connective tissue under the skin, the method comprising applying, particularly topically or intra-dermally applying, the peptide as defined in claim 1 to the subject.

20. A method for treating a condition caused or associated to the accumulation of fat in an internal organ, the method comprising administering a therapeutically effective amount of a peptide as defined in claim 1 to a subject in need thereof.

21. A cosmetic method for improving the bodily appearance of a mammal with subcutaneous fat herniated or accumulated within the fibrous connective tissue under the skin, the method comprising applying, particularly topically or intra-dermally applying, the cosmetic composition as defined in claim 10 to the subject.

22. A method for treating a condition caused or associated to the accumulation of fat in an internal organ, the method comprising administering a therapeutically effective amount of the pharmaceutical composition as defined in claim 15 to a subject in need thereof.


SEQUENCE LISTING (87 sequences)

SEQ ID NO: 1 - protein, 10 aa, synthetic construct
  REGION 1..10: note = peptide
  VARIANT 1: note = Can be deleted. It can be represented as (T)a, wherein a is 0 or 1.
  REGION 1..10: note = each one of the amino acids being L- or D-amino acid
  VARIANT 2: note = X represents (X1)b, X1 is W, N, Q or K, and b is 0 or 1
  VARIANT 4: note = X represents P, N or a basic amino acid
  VARIANT 7: note = X represents (X3)c, X3 is A, V, L or I, and c is 0 or 1
  VARIANT 8: note = X represents (X4)d, X4 is A, V, L or I, and d is 0 or 1
  VARIANT 9: note = Can be deleted. It can be represented as (F)e, wherein e is 0 or 1
  VARIANT 10: note = Can be deleted. It can be represented as (Y)f, wherein f is 0 or 1
TXDXEGXXFY

SEQ ID NO: 2 - protein, 10 aa, synthetic construct
  REGION 1..10: note = peptide
TWDPEGVIFY

SEQ ID NO: 3 - protein, 10 aa, synthetic construct
  REGION 1..10: note = peptide
  SITE 1..10: note = all D-amino acids
TWDPEGVIFY

SEQ ID NO: 4 - protein, 10 aa, synthetic construct
  REGION 1..10: note = peptide
  SITE 1: note = Acetylation - the N-terminal is -NHC(O)CH3
  SITE 1..10: note = all D-amino acids
TWDPEGVIFY

SEQ ID NO: 5 - protein, 10 aa, synthetic construct
  REGION 1..10: note = peptide
  SITE 1..10: note = all D-amino acids
  MOD_RES 10: note = AMIDATION, C-terminus is CONH2
TWDPEGVIFY

SEQ ID NO: 6 - protein, 10 aa, synthetic construct
  REGION 1..10: note = peptide
  MOD_RES 1: note = Acetylation - the N-terminus is -NHC(O)CH3
  SITE 1..10: note = all D-amino acids
  MOD_RES 10: note = AMIDATION
TWDPEGVIFY

SEQ ID NO: 7 - protein, 9 aa, synthetic construct
  REGION 1..9: note = peptide
  SITE 1..9: note = all D-amino acids
WDPEGVIFY

SEQ ID NO: 8 - protein, 9 aa, synthetic construct
  REGION 1..9: note = peptide
  SITE 1..9: note = all D-amino acids
TDPEGVIFY

SEQ ID NO: 9 - protein, 9 aa, synthetic construct
  REGION 1..9: note = peptide
  SITE 1..9: note = all D-amino acids
TWPEGVIFY

SEQ ID NO: 10 - protein, 9 aa, synthetic construct
  REGION 1..9: note = peptide
  SITE 1..9: note = all D-amino acids
TWDEGVIFY

SEQ ID NO: 11 - protein, 9 aa, synthetic construct
  REGION 1..9: note = peptide
  SITE 1..9: note = all D-amino acids
TWDPGVIFY

SEQ ID NO: 12 - protein, 9 aa, synthetic construct
  REGION 1..9: note = peptide
  SITE 1..9: note = all D-amino acids
TWDPEVIFY

SEQ ID NO: 13 - protein, 9 aa, synthetic construct
  REGION 1..9: note = peptide
  SITE 1..9: note = all D-amino acids
TWDPEGIFY

SEQ ID NO: 14 - protein, 9 aa, synthetic construct
  REGION 1..9: note = peptide
  SITE 1..9: note = all D-amino acids
TWDPEGVFY

SEQ ID NO: 15 - protein, 9 aa, synthetic construct
  REGION 1..9: note = peptide
  SITE 1..9: note = all D-amino acids
TWDPEGVIY

SEQ ID NO: 16 - protein, 9 aa, synthetic construct
  REGION 1..9: note = peptide
  SITE 1..9: note = all D-amino acids
TWDPEGVIF

SEQ ID NO: 17 - protein, 8 aa, synthetic construct
  REGION 1..8: note = peptide
  SITE 1..8: note = all D-amino acids
DPEGVIFY

SEQ ID NO: 18 - protein, 8 aa, synthetic construct
  REGION 1..8: note = peptide
  SITE 1..8: note = all D-amino acids
WDPEGVIY

SEQ ID NO: 19 - protein, 8 aa, synthetic construct
  REGION 1..8: note = peptide
  SITE 1..8: note = all D-amino acids
WDPEGVIF

SEQ ID NO: 20 - protein, 8 aa, synthetic construct
  REGION 1..8: note = peptide
  SITE 1..8: note = all D-amino acids
TDPEGVIY

SEQ ID NO: 21 - protein, 8 aa, synthetic construct
  REGION 1..8: note = peptide
  SITE 1..8: note = all D-amino acids
TDPEGVIF

SEQ ID NO: 22 - protein, 8 aa, synthetic construct
  REGION 1..8: note = peptide
  SITE 1..8: note = all D-amino acids
TWDPEGVI

SEQ ID NO: 23 - protein, 7 aa, synthetic construct
  REGION 1..7: note = peptide
  SITE 1..7: note = all D-amino acids
DPEGVFY

SEQ ID NO: 24 - protein, 7 aa, synthetic construct
  REGION 1..7: note = peptide
  SITE 1..7: note = all D-amino acids
DPEGVIY

SEQ ID NO: 25 - protein, 7 aa, synthetic construct
  REGION 1..7: note = peptide
  SITE 1..7: note = all D-amino acids
DPEGVIF

SEQ ID NO: 26 - protein, 7 aa, synthetic construct
  REGION 1..7: note = peptide
  SITE 1..7: note = all D-amino acids
WDPEGVI

SEQ ID NO: 27 - protein, 7 aa, synthetic construct
  REGION 1..7: note = peptide
  SITE 1..7: note = all D-amino acids
WDPEGVY

SEQ ID NO: 28 - protein, 7 aa, synthetic construct
  REGION 1..7: note = peptide
  SITE 1..7: note = all D-amino acids
WDPEGVF

SEQ ID NO: 29 - protein, 6 aa, synthetic construct
  REGION 1..6: note = peptide
  SITE 1..6: note = all D-amino acids
DPEGVI

SEQ ID NO: 30 - protein, 6 aa, synthetic construct
  REGION 1..6: note = peptide
  SITE 1..6: note = all D-amino acids
WDPEGV

SEQ ID NO: 31 - protein, 6 aa, synthetic construct
  REGION 1..6: note = peptide
  SITE 1..6: note = all D-amino acids
TWDPEG

SEQ ID NO: 32 - protein, 5 aa, synthetic construct
  REGION 1..5: note = peptide
  SITE 1..5: note = all D-amino acids
WDPEG

SEQ ID NO: 33 - protein, 7 aa, synthetic construct
  REGION 1..7: note = peptide
  SITE 1..7: note = all D-amino acids
WDPEGLY

SEQ ID NO: 34 - protein, 7 aa, synthetic construct
  REGION 1..7: note = peptide
  SITE 1..7: note = all D-amino acids
DPEGLFY

SEQ ID NO: 35 - protein, 7 aa, synthetic construct
  REGION 1..7: note = peptide
  SITE 1..7: note = all D-amino acids
DPEGVLY

SEQ ID NO: 36 - protein, 7 aa, synthetic construct
  REGION 1..7: note = peptide
  SITE 1..6: note = all D-amino acids
WDNEGLY

SEQ ID NO: 37 - protein, 7 aa, synthetic construct
  REGION 1..7: note = peptide
  SITE 1..7: note = all D-amino acids
NDPEGLY

SEQ ID NO: 38 - protein, 8 aa, synthetic construct
  REGION 1..8: note = peptide
  SITE 1..8: note = all D-amino acids
NDPEGVIY

SEQ ID NO: 39 - protein, 10 aa, synthetic construct
  REGION 1..10: note = peptide
  REGION 1..10: note = each one of the amino acids is L- or D-amino acid, provided that at least one of the amino acids is D-amino acid
TWDPEGVIFY

SEQ ID NO: 40 - protein, 10 aa, synthetic construct
  REGION 1..10: note = peptide
  MOD_RES 1: note = Acetylation - the N-terminal is -NHC(O)CH3
  REGION 1..10: note = each one of the amino acids is L- or D-amino acid, provided that at least one of the amino acids is D-amino acid
TWDPEGVIFY

SEQ ID NO: 41 - protein, 10 aa, synthetic construct
  REGION 1..10: note = peptide
  REGION 1..10: note = each one of the amino acids is L- or D-amino acid, provided that at least one of the amino acids is D-amino acid
  MOD_RES 1..10: note = AMIDATION
TWDPEGVIFY

SEQ ID NO: 42 - protein, 10 aa, synthetic construct
  REGION 1..10: note = peptide
  MOD_RES 1: note = Acetylation - the N-terminal is -NHC(O)CH3
  REGION 1..10: note = each one of the amino acids is L- or D-amino acid, provided that at least one of the amino acids is D-amino acid
  MOD_RES 1..10: note = AMIDATION
TWDPEGVIFY

SEQ ID NO: 43 - protein, 9 aa, synthetic construct
  REGION 1..9: note = peptide
  REGION 1..9: note = each one of the amino acids is L- or D-amino acid
TWPEGVIFY

SEQ ID NO: 44 - protein, 9 aa, synthetic construct
  REGION 1..9: note = peptide
  REGION 1..9: note = each one of the amino acids is L- or D-amino acid
TWDEGVIFY

SEQ ID NO: 45 - protein, 9 aa, synthetic construct
  REGION 1..9: note = peptide
  REGION 1..9: note = each one of the amino acids is L- or D-amino acid
TWDPGVIFY

SEQ ID NO: 46 - protein, 9 aa, synthetic construct
  REGION 1..9: note = peptide
  REGION 1..9: note = each one of the amino acids is L- or D-amino acid
TWDPEVIFY

SEQ ID NO: 47 - protein, 10 aa, synthetic construct
  REGION 1..10: note = peptide
  VARIANT 1: note = X represents (T)a, wherein a is 0 or 1
  REGION 1..10: note = the amino acids are L- or D-amino acids
  VARIANT 2: note = X represents (X1)b; X1 represents W or N, and b is 0 or 1
  VARIANT 8: note = X represents (X4)d; X4 represents I or L, and d is 0 or 1
  VARIANT 9: note = X represents (F)e; wherein e represents 0 or 1
  VARIANT 10: note = X represents (Y)f; wherein f represents 0 or 1
XXDPEGVXXX

SEQ ID NO: 48 - protein, 9 aa, synthetic construct
  REGION 1..9: note = peptide
  VARIANT 1: note = X represents (T)a, wherein a is 0 or 1
  REGION 1..9: note = the amino acids are L- or D-amino acids
  VARIANT 2: note = X represents (X1)b; X1 represents W or N; and b is 0 or 1
  VARIANT 7: note = X represents I or L
  VARIANT 8: note = X represents (F)e, wherein e is 0 or 1
  VARIANT 9: note = X represents (Y)f, wherein f is 0 or 1
XXDPEGXXX

SEQ ID NO: 49 - protein, 9 aa, synthetic construct
  REGION 1..9: note = peptide
  VARIANT 1: note = X represents (T)a, wherein a is 0 or 1
  REGION 1..9: note = the amino acids are L- or D-amino acids
  VARIANT 2: note = X represents (W)b, wherein b is 0 or 1
  VARIANT 7: note = X represents I or L
  VARIANT 8: note = X represents (F)e, wherein e is 0 or 1
  VARIANT 9: note = X represents (Y)f, wherein f is 0 or 1
XXDNEGXXX

SEQ ID NO: 50 - protein, 10 aa, synthetic construct
  REGION 1..10: note = peptide
  VARIANT 1: note = X represents (T)a, wherein a is 0 or 1
  SITE 1..10: note = all the amino acids are D-amino acids
  VARIANT 2: note = X represents (X1)b; wherein X1 is W or N; and b is 0 or 1
  VARIANT 8: note = X represents (X4)d; wherein X4 is I or L, and d is 0 or 1
  VARIANT 9: note = X represents (F)e, wherein e is 0 or 1
  VARIANT 10: note = X represents (Y)f, wherein f is 0 or 1
XXDPEGVXXX

SEQ ID NO: 51 - protein, 9 aa, synthetic construct
  REGION 1..9: note = peptide
  VARIANT 1: note = X represents (T)a, wherein a is 0 or 1
  SITE 1..9: note = all the amino acids are D-amino acids
  VARIANT 2: note = X represents (X1)b; wherein X1 is W or N, and b is 0 or 1
  VARIANT 7: note = X represents I or L
  VARIANT 8: note = X represents (F)e, wherein e is 0 or 1
  VARIANT 9: note = X represents (Y)f, wherein f is 0 or 1
XXDPEGXXX

SEQ ID NO: 52 - protein, 9 aa, synthetic construct
  REGION 1..9: note = peptide
  VARIANT 1: note = X represents (T)a, wherein a is 0 or 1
  SITE 1..9: note = all the amino acids are D-amino acids
  VARIANT 2: note = X represents (W)b, wherein b is 0 or 1
  VARIANT 7: note = X represents I or L
  VARIANT 8: note = X represents (F)e, wherein e is 0 or 1
  VARIANT 9: note = X represents (Y)f, wherein f is 0 or 1
XXDNEGXXX

SEQ ID NO: 53 - protein, 6 aa, synthetic construct
  SITE 1: note = Acetylation - the N-terminal end is -NHC(O)CH3
  MOD_RES 6: note = AMIDATION
SIYVAT

SEQ ID NO: 54 - protein, 10 aa, synthetic construct
  VARIANT 1: note = residue can be deleted. It can be represent as (T)a wherein a is 0 or 1.
  VARIANT 2: note = Can be W, N, Q or K and can be deleted. It can be represented as (X1)b, wherein X1 is W, N, Q or K, and b is 0 or 1.
  VARIANT 4: note = Can be P, N, a basic amino acid or a hydrophobic amino acid, and can be deleted. It can be represented as (X2)g, wherein X2 is P, N, a basic amino acid or a hydrophobic amino acid, and g is 0 or 1.
  VARIANT 7: note = Can be A, V, L or I and can be deleted. It can be represented as (X3)c, wherein X3 is A, V, L or I and c is 0 or 1.
  VARIANT 8: note = can be A, V, L or I and can be deleted. It can be represented as (X4)d, wherein X4 is A, V, L or I and d is 0 or 1.
  VARIANT 9: note = can be F or P and can be deleted. It can be represented as (X5)e wherein X5 is F or P and e is 0 or 1.
  VARIANT 10: note = can be deleted. It can be represented as (Y)f, wherein f is 0 or 1.
  REGION 1..10: note = Each amino acid can be an L-amino acid or a D-amino acid
TXDXEGXXXY

SEQ ID NO: 55 - protein, 7 aa, Synthetic construct
  SITE 1..7: note = Each amino acid is a D-amino acid
NDVEGLY

SEQ ID NO: 56 - protein, 7 aa, synthetic construct
  SITE 1..7: note = Each amino acid is a D-amino acid
NDAEGLY

SEQ ID NO: 57 - protein, 7 aa, synthetic construct
  SITE 1..7: note = Each amino acid is a D-amino acid
NDLEGVY

SEQ ID NO: 58 - protein, 7 aa, synthetic construct
  SITE 1..7: note = Each amino acid is a D-amino acid
NDLEGAY

SEQ ID NO: 59 - protein, 7 aa, synthetic construct
  SITE 1..7: note = Each amino acid is a D-amino acid
NDLEGPY

SEQ ID NO: 60 - protein, 7 aa, synthetic construct
  SITE 1..7: note = Each amino acid is a D-amino acid
NDEGLFY

SEQ ID NO: 61 - protein, 6 aa, synthetic construct
  SITE 1..6: note = Each amino acid is a D-amino acid
NDPEGY

SEQ ID NO: 62 - protein, 7 aa, synthetic construct
  SITE 1..7: note = Each amino acid is a D-amino acid
NDLEGLY

SEQ ID NO: 63 - protein, 6 aa, synthetic construct
  SITE 1..6: note = Each amino acid is a D-amino acid
NDEGLY

SEQ ID NO: 64 - protein, 6 aa, synthetic construct
  SITE 1..6: note = Each amino acid is a D-amino acid
NDEGLY

SEQ ID NO: 65 - protein, 6 aa, synthetic construct
  SITE 1..6: note = Each amino acid is a D-amino acid
NDEGLY

SEQ ID NO: 66 - protein, 7 aa, synthetic construct
  MOD_RES 1: note = Acetylation - the N-terminus is -NHC(O)CH3
  SITE 1..7: note = Each amino acid is a D-amino acid
NDPEGLY

SEQ ID NO: 67 - protein, 7 aa, synthetic construct
  MOD_RES 1: note = Acetylation - the N-terminus is -NHC(O)CH3
  SITE 1: note = Each amino acid is a D-amino acid
NDLEGLY

SEQ ID NO: 68 - protein, 7 aa, synthetic construct
  MOD_RES 1: note = Acylation - N terminus is CH3CH2CONH
  SITE 1..7: note = Each amino acid is a D-amino acid
NDLEGLY

SEQ ID NO: 69 - protein, 7 aa, synthetic construct
  MOD_RES 1: note = Acetylation - the N-terminus is -NHC(O)CH3
  MOD_RES 7: note = Amidation - the C-terminus is CONH2
  SITE 1..7: note = Each amino acid is a D-amino acid
NDPEGLY

SEQ ID NO: 70 - protein, 7 aa, synthetic construct
  MOD_RES 1: note = Acylation - the N-terminus is CH3(CH2)14CONH
  SITE 1..7: note = Each amino acid is a D-amino acid
NDLEGLY

SEQ ID NO: 71 - protein, 7 aa, synthetic construct
  MOD_RES 1: note = Acetylation - the N-terminus is CH3CONH
  MOD_RES 7: note = Amidation - The C-terminus is CONH2
  SITE 1..7: note = Each amino acid is a D-amino acid
NDLEGLY

SEQ ID NO: 72 - protein, 8 aa, synthetic construct
  VARIANT 3: note = Can be V, A, L, or P and can be deleted. It can be represented as (X2)g, wherein X2 is V, A, L, or P and g is 0 or 1.
  VARIANT 6: note = can be L, V or A and can be deleted. It can be represented as (X3)c wherein X3 is L, V or A, and c is 0 or 1.
  VARIANT 7: note = can be P or F and can be deleted. It can be represented as (X5)e, wherein X5 is P or F and e is 0 or 1.
  REGION 1..8: note = Each amino acid can be an L-amino acid or a D-amino acid.
NDXEGXXY

SEQ ID NO: 73 - protein, 7 aa, synthetic construct
  VARIANT 6: note = Can be deleted. It can be represented as (L)c wherein c is 0 or 1.
  REGION 1..7: note = Each amino acid is an L-amino acid or a D-amino acid.
NDPEGLY

SEQ ID NO: 74 - skipped

SEQ ID NO: 75 - protein, 7 aa, synthetic construct
  VARIANT 3: note = Can be P or L and can be deleted. It can be represented as (X2)g wherein X2 is P or L and g is 0 or 1.
  REGION 1..7: note = Each amino acid is an L-amino acid or a D-amino acid.
NDXEGLY

SEQ ID NO: 76 - protein, 8 aa, synthetic construct
  VARIANT 3: note = Can be V, A, L or P and can be deleted. It can be represented as (X2)g wherein X2 is V, A, L or P and g is 0 or 1.
  VARIANT 6: note = Can be L, V or A and can be deleted. It can be represented as (X3)c, wherein X3 is L, V or A and c is 0 or 1.
  VARIANT 7: note = Can be P or F and can be deleted. It can be represented as (X5)e wherein X5 is P or F and e is 0 or 1.
  SITE 1..8: note = Each amino acid is a D-amino acid
NDXEGXXY

SEQ ID NO: 77 - protein, 7 aa, synthetic construct
  VARIANT 6: note = Can be deleted. It can be represented as (L)c wherein c is 0 or 1.
  SITE 1..7: note = Each amino acid is a D-amino acid
NDPEGLY

SEQ ID NO: 78 - skipped

SEQ ID NO: 79 - protein, 7 aa, synthetic construct
  VARIANT 3: note = Can be P or L and can be deleted. It can be represented as (X2)g wherein X2 is P or L and g is 0 or 1.
  SITE 1..7: note = Each amino acid is a D-amino acid
NDXEGLY

SEQ ID NO: 80 - protein, 7 aa, synthetic construct
NDPEGLY

SEQ ID NO: 81 - protein, 7 aa, synthetic construct
  MOD_RES 1: note = Acetylation - The N-terminus is -NHC(O)CH3
NDPEGLY

SEQ ID NO: 82 - protein, 7 aa, synthetic construct
  SITE 1: note = D-Asparagine
NDPEGLY

SEQ ID NO: 83 - protein, 7 aa, synthetic construct
  SITE 2: note = D-Aspartic Acid
NDPEGLY

SEQ ID NO: 84 - protein, 7 aa, synthetic construct
  SITE 3: note = D-Proline
NDPEGLY

SEQ ID NO: 85 - protein, 7 aa, synthetic construct
  SITE 4: note = D-Glutamic Acid
NDPEGLY

SEQ ID NO: 86 - protein, 7 aa, synthetic construct
  SITE 6: note = D-Leucine
NDPEGLY

SEQ ID NO: 87 - protein, 7 aa, synthetic construct
  SITE 7: note = D-Tyrosine
NDPEGLY